(19) United States
(12) Patent Application Publication
(10) Pub. No.: US 2024/0293474 A1
(43) Pub. Date: Sep. 5, 2024

(54) Programmable Nanoencapsulation for Delivery of Probiotics in Vivo

(71) Applicant: The Trustees of Columbia University in the City of New York, New York, NY (US)

(72) Inventors: Tal DANINO, New York, NY (US); Kam LEONG, New York, NY (US); Tetsuhiro HARIMOTO, New York, NY (US); Jaeseung HAHN, New York, NY (US)

(73) Assignee: The Trustees of Columbia University in the City of New York, New York, NY (US)

(21) Appl. No.: 18/498,383

(22) Filed: Oct. 31, 2023

Related U.S. Application Data
(63) Continuation of application No. PCT/US22/28977, filed on May 12, 2022.
(60) Provisional application No. 63/187,556, filed on May 12, 2021.
(60) Provisional application No. 63/228,343, filed on Aug. 2, 2021.

(51) Int. Cl. A61K 35/74; A61K 35/00; A61P 35/00; C07K 14/195; C12N 15/70

(52) U.S. Cl. CPC A61K 35/74; A61P 35/00; C07K 14/195; C12N 15/70; A61K 2035/115; C12N 2830/002

(57) ABSTRACT

Programmable bacterial cells that comprise a gene that regulates capsular polysaccharide nanoencapsulation of the bacterium linked to an exogenous promoter, wherein expression of the gene and the nanoencapsulation can be programmed or controlled by an external modulator of the exogenous promoter and related compositions and methods.

### CROSS-REFERENCE TO RELATED APPLICATIONS

This is a continuation of International Patent Application No. PCT/US2022/028977 filed May 12, 2022, which claims the benefit of U.S. Provisional Patent Application Nos. 63/187,556 filed May 12, 2021, and 63/228,343 filed Aug. 2, 2021, each of which are hereby incorporated by reference in their entirety.

### STATEMENT REGARDING FEDERALLY SPONSORED RESEARCH OR DEVELOPMENT

This invention was made with government support LC160314 and BC160541 awarded by the Department of Defense and R01CA249160, U01CA197649, F99CA253756 and U01CA247573 awarded by the National Institutes of Health. The government has certain rights in the invention.

### TECHNICAL FIELD OF THE INVENTION

This disclosure generally relates to the fields of medicine and microbiology. More specifically, the disclosure relates to programmable bacteria cells (e.g., E. coli Nissle 1917 bacteria) that possess a genetically encoded microbial encapsulation system with tunable and dynamic expression of surface capsular polysaccharides to enhance therapeutic delivery, as well as related compositions and methods.

### BACKGROUND OF THE INVENTION

The microbiome plays numerous functional roles in human health and subsequently has led to focused interest in the use of live bacteria to treat disease. Since microbes can be engineered as intelligent living medicines that sense and respond to environments, they can colonize niches in the gastrointestinal tract, mouth, skin, lung, and tumors, and locally deliver therapeutics.

However, host toxicity from live bacteria has been shown to limit tolerated dose and efficacy, in some cases leading to termination of clinical trials. Moreover, unlike conventional drug carriers, the unique abilities of bacteria to continuously proliferate, chemotax, and produce therapeutic payloads in disease sites necessitates robust and temporal control of bacterial pharmacokinetics in vivo.

One approach to circumvent toxicity is the generation of genetic knockouts of immunogenic bacterial surface antigens such as lipopolysaccharide (LPS), but this strategy can result in permanent strain attenuation and reduced colonization, as seen in clinical trials of bacteria cancer therapy. Surface modulation has been widely utilized in cloaking drug delivery vehicles, and thus an alternative strategy is the synthetic coating of microbial surfaces with molecules such as alginate, chitosan, polydopamine, lipids, and nanoparticles. However, these one-time, static modifications of bacteria do not allow for in situ modulation and can lead to uncontrolled growth, off-target toxicity, or compromised cellular function resulting in reduced efficacy.

Accordingly, new designs are needed that facilitate precise control over bacterial immunogenicity and survivability in vivo, enabling novel drug delivery strategies such as enhanced dosing and in situ trafficking to maximize therapeutic efficacy and safety.

### BRIEF SUMMARY OF THE INVENTION

The present disclosure relates to programmable bacterial cells that comprise a gene that regulates capsular polysaccharide nanoencapsulation of the bacterium linked to an exogenous promoter, wherein expression of the gene and the nanoencapsulation can be programmed or controlled by an external modulator of the exogenous promoter.

In some embodiments, the programmable bacterial cells belong to at least one genus selected from the group consisting of Salmonella, Escherichia, Firmicutes, Bacteroidetes, Lactobacillus, and Bifidobacteria. In some embodiments, the programmable bacterial cells belong to the genus Escherichia. In particular embodiments, the programmable bacterial cells are Escherichia coli Nissle (EcN) cells.

In some embodiments, the programmable bacterial cells comprise a gene that regulates capsular polysaccharide nanoencapsulation selected from the group consisting of kfi and kps genes. In some embodiments, the programmable bacterial cells comprise a gene that regulates capsular polysaccharide nanoencapsulation selected from the group consisting of kfiA, kfiB, kfiC, kfiD, kpsE, kpsD, kpsM, kpsT, kpsC, kpsS, kpsF and kpsU. In some embodiments, the programmable bacterial cells exhibit desirable properties when the gene that regulates capsular polysaccharide nanoencapsulation is expressed (e.g., resistance to host immune system responses) or not expressed (e.g., increased clearance from the host).

In some embodiments, the programmable bacterial cells comprise at least one plasmid comprising a nucleic acid sequence which encodes a therapeutic agent. In some embodiments, the therapeutic agent is theta-toxin and the at least one plasmid is (ColE1). In some embodiments, the programmable bacterial cells comprise a functional proteic toxin-antitoxin system (e.g., Axe/Txe).

In some embodiments, the programmable bacterial cells comprise a promoter regulated by an exogenous agent. In some embodiments, the programmable bacterial cells comprise a promoter that is sensitive or respond to a particular environmental or physiological condition. In some embodiments, the programmable bacterial cells comprise a promoter that is induced by bacterial molecules. In some embodiments, the exogenous promoter is lac, which is activated with isopropyl-b-D-thiogalactopyranoside (IPTG). In some embodiments, the exogenous promoter is quorum sensing (e.g., AHL-sensing plux1 promoter). In some embodiments, the exogenous promoter is sensitive to pH (e.g., pCadC).

The present disclosure also relates to methods of treating a cancer (or tumor) in a subject comprising administering a therapeutically effective amount of programmable bacterial cells described herein to the subject, wherein the programmable bacterial cells comprise a nucleic acid encoding a therapeutic agent described herein, which capable of treating the cancer. In some embodiments, the cancer is colorectal cancer. In some embodiments, the cancer is breast cancer.

The present disclosure also relates to methods of reducing the rate of proliferation of a tumor cell comprising delivering a programmable bacterial cell described herein to the tumor cell. The present disclosure also relates to methods of killing a tumor cell comprising delivering a programmable bacterial cell described herein to the tumor cell. In some embodiments, the tumor cell is a colorectal tumor cell. In some embodiments, the tumor cell is a breast cancer cell. In some embodiments, the programmable bacterial cell is delivered to a subject orally, intravenously, subcutaneously, or intratumorally.

In some embodiments, the programmable bacterial cells described herein may be administered to a subject or delivered to a tumor in the form of a pharmaceutical composition, which may comprise one or more pharmaceutically acceptable carriers, diluents, or excipients.

The present disclosure also relates to articles of manufacture useful for treating a colorectal tumor. In some embodiments, the articles of manufacture comprise a container comprising programmable bacterial cells described herein, or pharmaceutical compositions comprising the same, as well as instructional materials for using the same to treat a colorectal tumor. In some embodiments, the articles of manufacture are part of a kit that comprises a bacterial culture vessel and/or bacterial cell growth media.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description of the Invention. This summary is not intended to identify key features or essential features of the claimed subject matter, nor is it intended to be used as an aid in determining the scope of the claimed subject matter.

### BRIEF DESCRIPTION OF THE SEVERAL VIEWS OF THE DRAWINGS

FIGS. 1A-1C provide an illustration of the programmable capsular polysaccharides (CAP) system for control over bacterial encapsulation and in vivo delivery profiles. The biosynthetic pathway of bacterial CAP was engineered for tunable and dynamic surface modulation of the probiotic E. coli Nissle 1917 with synthetic gene circuits. The CAP system modulates bacterial immunogenicity and survivability in vivo. By balancing these factors, the programmable CAP system is capable of reducing toxicity related to systemic bacterial administration and enables inducible bacterial translocation between tumors.

FIGS. 2A-2B illustrate the survival and immunogenicity of E. coli and S. typhimurium strains. a. Bacterial survival in human whole blood. 10⁸ CFU/mL bacteria was incubated in the blood for 2 hours and plated on LB agar for CFU enumeration. Limit of Detection (LOD) at 2×10² CFU/mL. All error bars represent standard error of mean (SEM) over three independent samples. b, Immune recognition of the bacteria. 5×10⁶ bacteria were systemically delivered to BALB/c mice by i.v. injection, and cheek blood was collected at 0.5-hour post injection (p.i.). TNFα levels were measured using ELISA. All error bars represent SEM (n=2 per group for EcN, K1, and VNP2009 groups; n=3 for MG1655 group)

FIGS. 3A-3D show how the sRNA knockdown screen identifies key genes in capsular polysaccharides (CAP) biosynthesis. a. Schematics of K5 CAP biosynthesis in EcN. CAP is composed of an alternating polymer chain of GlcA and GlcNAc connected to a poly-KDO linker. Subsequently CAP is transported from the inner bacteria membrane to the outer membrane. b. Quantification of microbial growth parameters of EcN knockdown (KD) strains in nutrient, blood, or phage containing media. Growth rate denotes maximum specific growth rate (hour-1) obtained by fitting growth curve to measured OD₆₀₀ over time. Blood viability is defined as fraction of bacterial CFU after 6 hours incubation in human blood over inoculated bacterial CFU. Phage sensitivity is calculated by area under the curve of bacterial turbidity over 6 hours of incubation with LB media containing ΦK1-5. c. Phage sensitivity of EcN and EcN ΔkfiC. Plaque forming assay demonstrates complete absence of infection and lysis in EcN ΔkfiC. The representative images show the difference between serially-diluted plaque forming units (PFU) of bacteria with and without CAP. d. TEM images showing CAP encapsulation of the cellular outer surface. kfiC knockout results in the absence of CAP nanostructure on the cell surface of EcN ΔkfiC. White arrows indicate cell surface.

FIGS. 4A-4F show the characterization of sRNA knockdown (KD) and knockout (KO) strains. a. Growth kinetics of KD strains of E. coli Nissle 1917 (EcN) in LB media. OD600 was measured over time in a plate reader. b. KD strain survival in human blood. Bacteria were inoculated in human whole blood for 0.5 hour, and plated on LB agar for CFU enumeration. c. Growth of KD strains in LB media containing ΦK1-5. WT strain without ΦK1-5 was included as a baseline bacterial growth. d. Growth of KO strains in LB media containing ΦK1-5. WT strain without ΦK1-5 was included as a baseline bacterial growth. (n=3 per group. All error bars represent SEM.) e-f KO strain survival in human blood. Bacteria were inoculated in human whole blood for 0.5 hour (e) or 6 hours (f) and plated on LB agar for CFU enumeration. All ‘n’ denotes number of biological replicates.

FIGS. 5A-5C illustrate capsular polysaccharide thickness quantification. a. Histograms of the thickness of polysaccharide layer of EcN and EcN ΔkfiC. The Gaussian curve were fitted to obtain mean and standard deviation of the polysaccharide layer of each strain. b. SDS-PAGE gel of CAP extracted from EcN and EcN ΔkfiC. Alcian blue stain confirmed presence of ˜180 kDa band for EcN. M=Precision Plus Protein PRECISION PLUS PROTEIN™ All Blue Standards (Biorad). This experiment was repeated twice. Source data are provided at the end of this document. c. TEM image processing of polysaccharide layer. Raw images were first processed using Gaussian blur to reduce noise and further transformed into binary images for image analysis to measure the thickness of ruthenium red-stained polysaccharide layer.

FIGS. 6A-6I show bacterial protection against environmental threats. a-g. Growth kinetics of EcN and EcN ΔkfiC in LB media containing sublethal concentration of antibiotics. (a) No antibiotics, (b) 10 μg/mL Spectinomycin, (c) 2 μg/mL Ampicillin, (d) 0.2 μg/mL Tetracycline. (e) 1 μg/mL Gentamicin, (f) 10 μg/mL Kanamycin, (g) 5 μg/mL Streptomycin (n.s.=0.0585, **P=0.0043, **P=0.0042, n.s.=0.255, *P=0.041, **P=0.0089, *P=0.016 respectively. n=3. two-way ANOVA). h. Bacterial survival in low pH condition. Bacteria were incubated in LB media at pH=2.5 for 1 hour and plated on LB agar for CFU enumeration (****P<0.0001, n=3 per group. Unpaired t-test.). pH was adjusted using hydrochloric acid. i. Bacterial survival in human blood. Bacteria were inoculated in human whole blood for 0.5 hours and plated on LB agar for CFU enumeration (***P<0.001, n=3 per group. Unpaired t-test.). All error bars represent SEM. All ‘n’ denotes number of biological replicates.

FIGS. 7A-7D show the characterization of CAP deletion in K1 and K5 strains. a,b. Phage sensitivity of WT and KO mutant of E. coli (a) K5 and (b) K1 strains. K1 CAP protects against T7 phage. and K5 CAP is targeted by ΦK1-5 phage. Quantification of K5 plaque assay is shown at the bottom bar plot. c. K1 bacterial survival in serum. WT and ΔneuC K1 strains were inoculated in mouse serum for 1.5 hour and plated on LB agar for CFU enumeration. All error bars represent SEM over two independent samples.

FIGS. 8A-8D show the design and characterization of the inducible capsular polysaccharides (iCAP) a lac promoter to allow inducible CAP expression via the small molecule IPTG. Copy number of the kfiC gene was modified to minimize basal kfiC expression. b. SDS-PAGE gel stained with Alcian blue showed elevating levels of CAP production corresponding to the IPTG concentration (top). The densitometric analysis of CAP bands demonstrated that CAP production reaches maximum at approximately 1 μM IPTG (bottom). c. SDS-page gels and densitometric analysis show CAP kinetics upon induction (left) and decay (right). d. Ruthenium red-stained TEM images showing change in CAP in titrating IPTG concentration. Histograms reveal shift in cellular outer layer thickness as IPTG concentration increases. Insets show representative images of bacteria and zoomed outer surface structure. Dotted lines indicate inner (white) and outer (gray) perimeters of CAP. Scale bar is 40 nm (left) or 200 nm (right) in each inset.

FIGS. 9A-9D show the phage sensitivity of the programmable capsular polysaccharide (iCAP) system. a. Growth curve of EcN expressing kfiC gene under various copy number plasmids. Bacteria were grown in LB media containing ΦK1-5. b. Phage plaque assay of induced and uninduced EcN iCAP. Absence of IPTG resulted in complete immunity against ΦK1-5. IPTG induction rescued sensitivity to ΦK1-5. c. Coincubation of EcN ΔkfiC transformed with plasmids encoding kfiC and lacI (referred to as EcN iCAP hereafter) and ΦK1-5 in varying concentrations of IPTG. Inversely proportional relationship between IPTG concentration and viability of EcN iCAP were observed. d. Growth curve of EcN iCAP in LB media containing ΦK1-5. iCAP was pre-induced in various IPTG concentrations. Upon inoculation, rapid bacteria lysis event was observed after 3.5 hours. Inversely proportional relationship between IPTG concentration and bacteria lysis were observed. All error bars represent SEM over three independent samples.

FIGS. 10A-10J illustrate the tunable interaction of the programmable capsular polysaccharides (CAP) system with host immune factors. a. Bacteria were encapsulated using the iCAP system and exposed to human whole blood to test CAP-mediated protection. Elevating levels of CAP activation with IPTG enabled a corresponding increase in bacterial survival in human whole blood. Bacteria were pre-induced with IPTG before blood exposure. b. Representative images of bacteria spotted on LB agar plate after 1-hour incubation in human whole blood (right). c. Survival kinetics using varying levels of IPTG induction before incubation with human whole blood. 102, 103 or 104 nM IPTG were added to the bacteria overnight culture to pre-induce the iCAP system. d. Induced or un-induced iCAP bacteria were co-cultured with BMDMs to test CAP-mediated protection from phagocytosis. e. BMDMs were lysed after incubating with bacteria for 30 minutes to enumerate phagocytosed bacteria (**P=0.007, two-sided unpaired t-test). f. Representative fluorescence microscopy images showing bacteria (GFP, top) in phagocytes (bright-field overlayed with GFP, bottom). Scale bars, 10 μm. g, Human THP-1 cells were co-incubated with EcN, EcN ΔkfiC or EcN iCAP (pre-induced with 10 μM IPTG) to test for immunogenicity. h-j, Cytokine levels in the culture media were measured using Luminex multiplex assay including TNFα (**P=0.001 and ***P=0.0002, respectively, at 24 hours after incubation), IL1β (*P=0.017 at 4 hours after incubation) and IL6 (*P=0.029 at 4 hours after incubation). All error bars represent s.e.m. over three independent samples, and statistical analyses were performed using one-way ANOVA with Tukey's multiple comparison test. LOD at 2×10² CFU/mL (b and c) and 1×10² CFU ml-1 (e).

FIGS. 11A-11C show bacterial survival in mouse whole blood. a, b. EcN and EcN ΔkfiC were inoculated in mouse whole blood for (a) 1 and (b) 2 hours and plated on LB agar for CFU enumeration. c. EcN iCAP were grown with and without 10 μM IPTG and were inoculated in mouse whole blood for 1 hour and plated on LB agar for CFU enumeration (***P=0.0005, **P=0.003, *P=0.0148. Two-sided unpaired t-test). All error bars represent SEM over three independent samples.

FIGS. 12A-12B show inducible protection from phagocytosis using iCAP. a. Histogram showing number of phagocytosed bacteria in murine bone marrow derived macrophages (BMDM). b. iCAP activation reduced levels of phagocytosis by BMDM (*P=0.037, two-sided unpaired t-test). Phagocytosis index=(% BMDM containing >1 bacterium)×(mean number of bacteria per BMDM). All error bars represent SEM over three independent samples.

FIGS. 13A-13D show cytokine production from THP-1 co-cultured with bacteria. a-d. Cytokine levels in the culture media were measured using Luminex multiplex assay including G-CSF (a. *P=0.01 at 24 hours post incubation), MIP1α (b. *P<0.02 and 0.01 at 4 hours post incubation), IL8 (c. *P=0.01 and **P=0.001, respectively, at 4 hours post incubation), and IL10 (d. *P=0.03 and 0.02 at 24 hours post incubation). All error bars represent standard error of mean (SEM) over three independent samples, and statistical analyses were performed using one-way ANOVA with Tukey's multiple comparison test.

FIG. 14 shows bacterial survival in human plasma. EcN and EcN ΔkfiC were inoculated in human plasma for 0.5 hour and plated on LB agar for CFU enumeration (*** P=0.0005, two-sided unpaired t-test). All error bars represent SEM over three independent samples.

FIGS. 15A-15N show that transient capsular polysaccharides (CAP) activation improves systemic bacterial delivery and efficacy in vivo. a. Host response was evaluated after bacterial administration in mice. EcN iCAP was pre-induced with 10 μM IPTG and allowed to gradually attenuate CAP over time. b-f. Serum cytokine levels after 5×10⁶ CFU bacterial administration. IL1β (b, *P=0.039 at 4 hours p.i.), IL6 (c, **P=0.0037 at 4 hours p.i.), IL10 (d, **P=0.0089 and *P=0.014, respectively, at 24 hours p.i.), G-CSF (e, ****P<0.0001 and **P<0.01, respectively, at 24 hours p.i.) and GM-CSF (f, *P=0.019 at 4 hours p.i.) were measured. g. Toxicity was evaluated by bacterial administration with elevating doses. h. Change in animal body weight after i.v. bacterial administration at 5×10⁶ CFU (**P=0.004; n=10, 5 and 5 mice for EcN iCAP, EcN and EcN ΔkfiC groups, respectively). i. Survival curve (>15% body weight reduction; n≥5 mice per group) after bacterial administration at 1˜7×10⁷ CFU. j. Dose-toxicity curve with MTD=4.4×10⁷ CFU, 5.8×10⁶ CFU and 9.6×10⁶ CFU for EcN iCAP, EcN and EcN ΔkfiC, respectively. MTD was calculated based on TD₅₀ (>10% body weight drops p.i.; non-linear regression with least squares fit; n≥5 per group). k. Mice bearing tumors were intravenously injected with EcN MTD, EcN ΔkfiC MTD and EcN iCAP MTD (pre-induced with 10 μM IPTG) or EcN iCAP low (pre-induced with 10 μM IPTG) at 5×10⁶, 1×10⁷, 5×10⁷ or 5×10⁶ CFU, respectively. Bacteria were engineered to produce TT. 1. Bacterial growth trajectories in subcutaneous CT26 tumors measured by bacterial luminescence (****P<0.0001; n=14, 13, 9 and 13 tumors for EcN MTD, EcN ΔkfiC MTD, EcN iCAP MTD and EcN iCAP low groups, respectively). Luminescence values are normalized to basal luminescence of individual strains. m, n. Therapeutic efficacy measured by relative tumor size over time in a syngeneic CT26 model (m; ****P<0.0001, ***P=0.0008, **P=0.003; n=14, 13, 9, 13 and 11 tumors for EcN MTD, EcN ΔkfiC MTD, EcN iCAP MTD, EcN iCAP low and PBS groups, respectively) and in a genetically engineered spontaneous breast cancer MMTV-PyMT mouse model (n; *P=0.0197; n=15, 15 and 9 tumors for EcN MTD, EcN iCAP MTD and PBS groups, respectively). MMTV-PyMT tumors were measured by calipering three orthotopic regions in mammary glands (upper left, upper right and bottom). Mice in PBS groups reached study endpoint 10 days p.i. Statistical analyses were performed using one-way ANOVA (b-f) and two-way ANOVA (h, i-n) with Tukey's multiple comparison test. Bacteria were engineered to produce TT (1-n). All error bars represent s.e.m. over three independent samples unless otherwise noted. All ‘n’ denotes number of biological replicates. a.u., arbitrary units; GM-CSF, granulocyte-macrophage colony-stimulating factor; p.i., post injection.

FIGS. 16A-16D show bacterial cytokine and neutrophil levels in blood after intravenous injection of bacteria. a. Bacterial count in circulation upon i.v. delivery. 5×10⁶ bacteria were systemically delivered to BALB/c mice. and cheek blood was collected. Viable bacteria were detected by plating on agar and counting CFU (*P=0.013, two-way ANOVA; n=5 per group). b. Change in TNFα levels after bacterial injection. Serum were collected and cytokine levels were measured using ELISA assay (***P=0.0007, two-way ANOVA with Sidak's multiple comparisons test; n=5 per group). c. Serum MKC levels measured using Luminex multiplex assay (*P=0.01 and 0.02, Oneway ANOVA with Tukey's multiple comparison test, 24 hours p.i.). d. Neutrophil count after bacterial i.v. injection (*P=0.04, two-way ANOVA with Sidak's multiple comparisons test; n=5 per group). EcN iCAP were pre-induced with 10 μM IPTG. Dotted line represents normal neutrophil count, taken from the mean of neutrophil count from untreated mice. By 12 days after bacterial administration, neutrophil level appeared to decrease back to normal level for all groups. All error bars represent SEM. All ‘n’ denotes number of biological rep ice in PBS groups reached study endpoint 10 days p.i.). All error bars represent SEM.

FIGS. 17A-17B shows the toxicity characterization of iCAP strains in sepsis model. a. 10⁶ CFU bacteria were intraperitoneally administered to BALB/c mice. EcN iCAP was pre-induced with 10 μM IPTG. iCAP group showed minimal drop in weight compared to EcN and EcN ΔkfiC groups. (*P=0.0153, two-way ANOVA with Turkey's multiple comparison test. n=5 mice per group). All error bars represent SEM. b. Survival curve after 107 CFU bacterial administration. Animals injected with EcN iCAP all survived while EcN group all succumbed within 2 days. All ‘n’ denotes number of biological replicates.

FIG. 18 shows the bacterial biodistribution upon intravenous delivery in vivo. BALB/c mice were intravenously administered with EcN, EcN ΔkfiC, or EcN iCAP. EcN iCAP was pre-induced with 10 μM IPTG. Spleen and liver were harvested after 1 day, homogenized. and spotted on LB-agar plate for CFU enumeration. Transient protection by EcN iCAP demonstrated reduced CFU in peripheral organs compared to EcN ΔkfiC. (****P<0.0001, *P=0.0452, two-way ANOVA with Turkey's multiple comparison test. LOD=3×10⁶ CFU/g). All error bars represent SEM over three independent samples.

FIG. 19 shows the change in animal body weight after bacterial administration at MTD in CT26 model. Mice were i.v. injected with EcN MTD (5×10⁶ CFU, n=8), EcN ΔkfiC MTD (1×10⁷ CFU, n=8), EcN iCAP MTD (pre-induced with 10 μM IPTG, 5×10⁷ CFU, n=4), EcN iCAP low (pre-induced with 10 μM IPTG, 5×10⁶ CFU, n=8) or PBS (n=7). All bacteria were engineered to produce TT. All animals showed similar drop in body weight at MTD (*P=0.03, n.s. P>0.49; two-way ANOVA with Turkey's multiple comparison test). All error bars represent SEM.

FIGS. 20A-20B shows the results of bacterial administration at MTD in PyMT MMTV model. a. Bacterial growth trajectories in PyMT tumors after intravenous delivery in vivo. Each line represents average of bacterial growth trajectories in tumors quantified by bacterial luminescence over time for each bacterial strain injected. Tumors injected with EcN iCAP MTD showed higher bacterial luminescence compared to tumor injected with EcN MTD (*P=0.044, Two-way ANOVA with Turkey's multiple comparison test; n=15 tumors for EcN MTD and EcN iCAP MTD groups). Luminescence values are normalized to basal luminescence of individual strains. All bacteria were engineered to produce TT. b. Change in animal body weight after bacterial administration at MTD. Mice were i.v. injected with EcN MTD or EcN iCAP MTD (pre-induced with 10 μM IPTG). Both groups showed similar drop in animal body weight (***P=0.0002, n.s. P>0.1; two-way ANOVA with Turkey's multiple comparison test; n=5, 7, 3 mice for EcN, EcN iCAP, and PBS groups, respectively). All error bars represent SEM. All ‘n’ denotes number of biological replicates.

FIGS. 21A-21B illustrate a bacterial pharmacokinetics model. a. A 3-compartment pharmacokinetic (PK) model for delivery via i.v. injection. To simulate the i.v. injection, initial conditions are set so that the bacterial population in each compartment other than blood is equal to zero. The magnitude of initial condition in blood acts as the different injection doses. b. A 4-compartment PK model for delivery via i.t. injection. To simulate the i.t. injection, the initial conditions are set so that the bacterial population in each compartment other than treated tumor is equal to zero.

FIG. 22 shows the phage sensitivity of intratumoral bacteria. BALB/c mice bearing subcutaneous CT26 tumors were intravenously administered with EcN, EcN ΔkfiC, or EcN iCAP at a dose of 5×10⁶ CFU. EcN iCAP was pre-induced with 10 μM IPTG. Intratumoral bacteria were isolated after 2 days from supernatants of the homogenized tumors. Bacteria were grown in LB media with and without ΦK1-5. EcN iCAP grew in the presence of ΦK1-5, indicating the loss of CAP. All error bars represent SEM over three independent samples. Background OD₆₀₀ was subtracted.

FIGS. 23A-23F show that in situ activation of the programmable capsular polysaccharides (CAP) enables bacterial translocation and drug delivery to distal tumors. a. Schematics of iCAP mediated bacterial translocation. EcN are injected into one tumor (treated tumor). iCAP activation enables bacteria translocation to distal tumors. b. Mice harboring multiple tumors are injected with EcN iCAP into a single tumor (treated tumor). Subsequently, mice were fed 10 mM IPTG water to activate iCAP in situ. Mice were imaged daily for bacterial bioluminescence to track tumor colonization in tumors. To quantify bacterial biodistribution, organs were harvested and bacterial colonies are counted after 3 days. c. Inducible translocation of EcN iCAP to distal tumors in CT26 syngeneic (left), 4T1 orthotopic (middle), and MMTV-PyMT genetically engineered (right) mouse tumor models. Representative IVIS images showing bacterial translocation in vivo. White arrows indicate location of bacterial injection. Black allows indicate location of bacterial translocation. Translocation is quantified by fraction of bacteria found in distal tumor compared to treated tumor. Bacteria number is measured by performing biodistribution for CFU/g enumeration. iCAP activation showed marked increase in bacterial translocation (*P=0.032, *P=0.029, **P=0.003, Mann-Whitney test). d. Representative images of ex vivo organ images taken with IVIS showing bacterial tumor translocation in 4T1 orthotopic mouse model. e. Schematics of engineered EcN capable of programmable translocation and therapeutic expression. Therapeutic production is externally controlled by an inducer AHL. The engineered EcN was injected into a single tumor and IPTG-induced to translocate to distal tumors, and AHL-induced to deliver therapeutics. f. Therapeutic efficacy in treated and distal CT26 tumors measured by relative tumor growth over time. Bacteria were injected into a single treated tumor. The translocation was controlled by IPTG water. 3 days p.i., AHL was administered to induce therapeutic expression. Distal tumor growth was suppressed only when therapeutic bacteria were able to translocate (n.s. P=0.83, **P=0.004, two-way ANOVA with Bonferroni posttest, n=6, 5 for both treated and distal tumors, respectively). All error bars represent SEM.

FIGS. 24A-24D illustrate the inducible translocation of EcN iCAP in the CT26 model. a. Inducible translocation of EcN iCAP from treated tumors to distal tumors in CFU. Mice bearing subcutaneous CT26 tumors were injected intratumorally with EcN iCAP to one tumor (treated). One group was fed with water containing IPTG 1 day p.i. (+IPTG in blue) to activate iCAP in situ. Top graphs represent bacterial CFU in tumors. Tumors were harvested after 3 days p.i., homogenized. and spotted on LB-agar plate for CFU enumeration. Bars denotes medians. Bottom graphs represent bacterial CFU connected with lines showing individual tumor pairs. b. Inducible translocation of EcN iCAP from treated tumors to distal tumors quantified by bacterial luminescence. Top graphs represent bacterial luminescence in tumors, corresponding to IVIS images. Tumors were harvested after 3 days p.i. and imaged ex vivo. Bars denotes medians. Bottom graphs represent bacterial luminescence connected with lines showing individual tumor pairs. c. Translocation of unmodified EcN from treated tumors to distal tumors quantified by bacterial luminescence. Top graphs represent bacterial luminescence in tumors, corresponding to IVIS images. Tumors were harvested after 3 days p.i. and imaged ex vivo. Bars denotes medians. Bottom graphs represent bacterial luminescence connected with lines showing individual tumor pairs. d. Bacterial biodistribution upon intratumoral administration and translocation in vivo. Tumors, spleen and liver were harvested after 3 days p.i., homogenized. and spotted on LB-agar plate for CFU enumeration. In situ induction of EcN iCAP demonstrated colonization of distal tumors. Bars denotes median. e. Change in animal body weight after intratumoral bacterial administration and induced translocation (n=5, 4 mice for +IPTG and −IPTG groups, respectively). Graphs represent % change in animal body weight p.i. All error bars represent SEM.

FIGS. 25A-25D show the individual bacteria growth trajectories in tumors after intratumoral administration of single tumor flank in vivo. a-c. Mice bearing either (a) subcutaneous CT26, (b) orthotropic 4T1, or (c) spontaneous PyMT-MMTV tumors were injected intratumorally with EcN iCAP to one tumor (treated. dotted lines). One group was fed with water containing IPTG 1 day p.i. (+IPTG, blue lines) to activate iCAP in situ. Graphs represent individual bacterial growth trajectories in tumors quantified by bacterial luminescence over time. corresponding to IVIS images. Increasing level of bacterial luminescence in untreated tumors (distal, solid lines) was observed in groups induced with IPTG. d. Mice bearing subcutaneous CT26 tumors were injected intratumorally with EcN to one tumor (treated. dotted lines). Graphs represent individual bacterial growth trajectories in tumors quantified by bacterial luminescence over time. corresponding to IVIS images. Increasing level of bacterial luminescence in untreated tumors (distal, solid lines) was observed.

FIGS. 26A-26C illustrates the inducible translocation of EcN iCAP in 4T1 model. a. Inducible translocation of EcN iCAP from treated tumors to distal tumors in CFU. Mice bearing orthotropic 4T1 tumors were injected intratumorally with EcN iCAP to one tumor (treated). One group was fed with water containing IPTG 1 day p.i. (+IPTG in blue) to activate iCAP in situ. Top graphs represent bacterial CFU in tumors. Tumors were harvested after 3 days p.i., homogenized. and spotted on LB-agar plate for CFU enumeration. Bars denotes medians. Bottom graphs represent bacterial CFU connected with lines showing individual tumor pairs. b. Inducible translocation of EcN iCAP from treated tumors to distal tumors quantified by bacterial luminescence. Top graphs represent bacterial luminescence in tumors, corresponding to IVIS images. Tumors were harvested after 3 days p.i. and imaged ex vivo. Bars denotes medians. Bottom graphs represent bacterial luminescence connected with lines showing individual tumor pairs. c. Bacterial biodistribution upon intratumoral administration and translocation in vivo. Tumors, spleen and liver were harvested after 3 days p.i., homogenized. and spotted on LB-agar plate for CFU enumeration. In situ induction of EcN iCAP demonstrated colonization of distal tumors. Bars denotes median. d. Change in animal body weight after intratumoral bacterial administration and induced translocation (n=4 mice for both +IPTG and −IPTG groups). Graphs represent % change in animal body weight p.i. All error bars represent SEM.

FIGS. 27A-27D illustrate the inducible translocation of EcN iCAP in PyMT-MMTV model. a. Inducible translocation of EcN iCAP from treated tumors to distal tumors in CFU. Mice bearing spontaneous PyMT-MMTV tumors were injected intratumorally with EcN iCAP to one tumor (treated). One group was fed with water containing IPTG 1 day p.i. (+IPTG in blue) to activate iCAP in situ. Top graphs represent bacterial CFU in tumors. Tumors were harvested after 3 days p.i., homogenized. and spotted on LB-agar plate for CFU enumeration. Bars denotes medians. Bottom graphs represent bacterial CFU connected with lines showing individual tumor pairs. b. Inducible translocation of EcN iCAP from treated tumors to distal tumors quantified by bacterial luminescence. Top graphs represent bacterial luminescence in tumors, corresponding to IVIS images. Tumors were harvested after 3 days p.i. and imaged ex vivo. Bars denotes medians. Bottom graphs represent bacterial luminescence connected with lines showing individual tumor pairs. c. Bacterial biodistribution upon intratumoral administration and translocation in vivo. Tumors, spleen, and liver were harvested after 3 days p.i., homogenized. and spotted on LB-agar plate for CFU enumeration. In situ induction of EcN iCAP demonstrated colonization of distal tumors. Bars denotes median. d. Change in animal body weight after intratumoral bacterial administration and induced translocation (n=4 for mice both +IPTG and −IPTG groups). Graphs represent % change in animal body weight p.i. All error bars represent SEM.

FIGS. 28A-28C show the individual growth trajectories of therapeutic bacteria in tumors after intratumoral administration of single tumor flank in vivo. a-c. Mice bearing subcutaneous CT26 tumors were injected intratumorally with EcN iCAP engineered to produce TT when induced with AHL to one tumor. (a) One group was fed with water containing IPTG 1 day p.i. to activate iCAP in situ, and subcutaneously injected with AHL to induce TT expression (+IPTG+AHL). (b) One group only received AHL (−IPTG+AHL). (c) One group only received IPTG (+IPTG−AHL). Graphs represent individual bacterial growth trajectories in tumors quantified by bacterial luminescence over time. corresponding to IVIS images. Increasing level of bacterial luminescence in untreated tumors (distal, solid lines) was observed in groups induced with IPTG.

FIG. 29 shows the treated and distal CT26 tumors from EcN iCAP control group measured by relative tumor growth over time. Bacteria were injected into a single treated tumor. The translocation was controlled by IPTG water. After 3 days of initial injection, AHL were administered to induce therapeutic expression. iCAP control does not contain theta-toxin gene. (n.s. P=0.92, two-way ANOVA with Bonferroni posttest, n=6 for both treated and distal tumors). All error bars represent standard error of mean (SEM). All ‘n’ denotes number of biological replicates.

FIG. 30 shows the change in animal body weight after intratumoral therapeutic bacterial administration and induced translocation. Mice bearing subcutaneous CT26 tumors were injected intratumorally with EcN iCAP engineered to produce TT when induced with AHL to one tumor. One group was fed with water containing IPTG 1-day p.i. (+IPTG−AHL, n=6) to activate iCAP in situ. One group was subcutaneously injected with AHL to induce TT expression (−IPTG+AHL, n=5). One group received both IPTG and AHL (+IPTG+AHL, n=6). Graphs represent % change in animal body weight p.i. All error bars represent SEM.

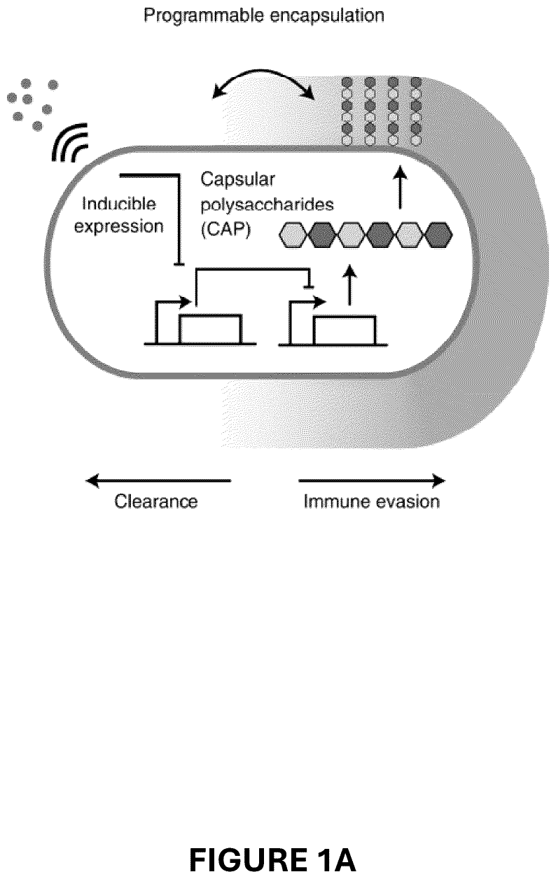

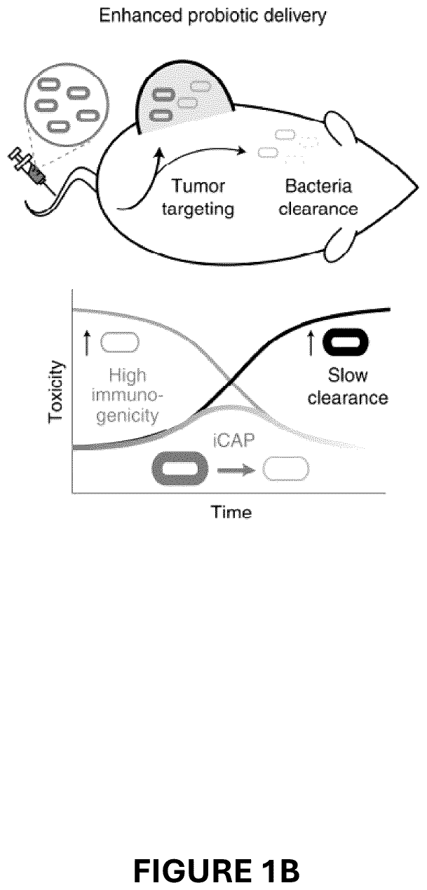

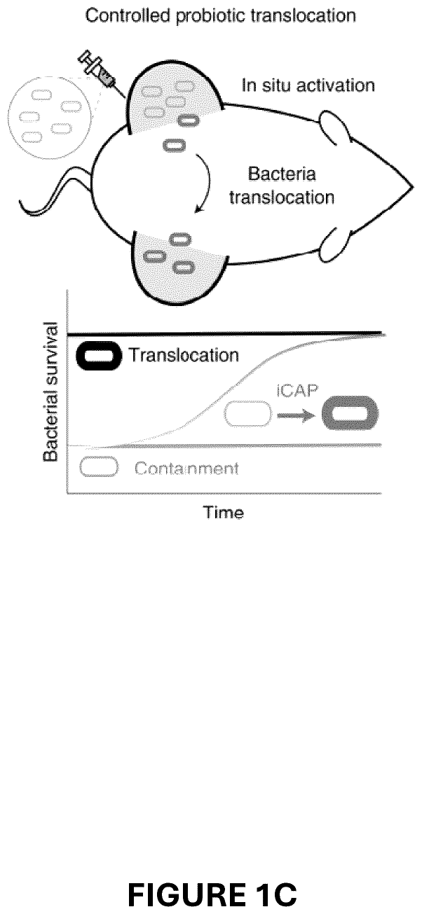

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

In addition, ranges provided in the specification and appended claims include both end points and all points between the end points. Therefore, a range of 1.0 to 2.0 includes 1.0, 2.0, and all points between 1.0 and 2.0.

The term “about” as used herein when referring to a measurable value such as an amount, a temporal duration, and the like, is meant to encompass variations of .+−0.20%, .+−0.10%, .+−0.5%, .+−0.1%, or .+−0.0.1% from the specified value, as such variations are appropriate to perform the disclosed methods.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or lists of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of “consisting essentially of,” when used in the claims, shall have its ordinary meaning as used in the field of patent law.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of and “consisting essentially of shall be closed or semi-closed transitional phrases, respectively.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

The inventions described herein relate to programmable bacterial cells that comprise a gene that regulates capsular polysaccharide nanoencapsulation of the bacterium linked to an exogenous promoter, wherein expression of the gene and the nanoencapsulation can be programmed or controlled by an external modulator of the exogenous promoter as described hereinbelow.

### Programmable Bacteria Cells

In some embodiments of the inventions described herein, programmable bacterial cells comprise engineered surface capsular polysaccharides (CAP). The programmable bacterial cells comprise heterologous nucleic acids that modulate the expression of CAP on the surface of the bacterial cells in response to the presence or absence of external modulators in the cells' environment, such as small molecules, bacterial cell population density, or pH.

The term “heterologous nucleic acid sequence” refers to a nucleic acid derived from a different organism that encodes for a protein and which has been recombinantly introduced into a cell, In some embodiments, the heterologous nucleic acid sequence is introduced by transformation in order to produce a recombinant bacterial cell. Methods for creating recombinant bacterial cells are well known to those of skill in the art. Such methods include, but are not limited to, different chemical, electrochemical and biological approaches, for example, heat shock transformation, electroporation, liposome-mediated transfection, DEAE-Dextran-mediated transfection, or calcium phosphate transfection. Multiple copies of the heterologous nucleic acid sequence (e.g., between 2 and 10,000 copies) may be introduced into the cell.

In some embodiments, the heterologous nucleic acid sequences are in a plasmid. In some embodiments, the heterologous nucleic acid sequences are in a single operon and are integrated into the genome of the programmable bacterial cells. In some embodiments, the programmable bacterial cells comprise at least one exogenous promoter that is in operable linkage with one or more of the heterologous nucleic acid sequences.

As used herein, the term “promoter” means at least a first nucleic acid sequence that regulates or mediates transcription of a second nucleic acid sequence through some manner of operable linkage. A promoter may comprise nucleic acid sequences near the start site of transcription that are required for proper function of the promoter. As an example, a TATA element for a promoter of polymerase II type. Promoters of the present inventions can include distal enhancer or repressor elements that may lie in positions from about 1 to about 500 base pairs, from about 1 to about 1,000 base pairs, from 1 to about 5,000 base pairs, or from about 1 to about 10,000 base pairs or more from the initiation site.

In embodiments of the invention, an “exogenous promoter” refers to a promotor originating from outside the programmable bacterial cell which mediates the transcription of one or more nucleic acids in the presence or absence of at least one external modulator. In some embodiments, the exogenous promoter mediates transcription of a nucleic acid sequence in the presence or absence of at least one, two, three, four, or five or more external modulator.

An “operable linkage” refers to an operative connection between nucleic acid sequences, such as for example between a control sequence (e.g., a promoter) and another nucleic acid sequence that codes for a protein i.e., a coding sequence. If a promoter can regulate transcription of an exogenous nucleic acid sequence, then it is in operable linkage with the gene.

In accordance with the purposes of the inventions described herein, the programmable bacterial cells are preferably non-pathogenic and colonize tumors. One of ordinary skill in the art would know how to attenuate pathogenic bacteria to create non-pathogenic bacteria. In some embodiments, the bacteria are attenuated by removing, knocking out, or mutating a virulence gene such as altering genetic components of the bacterial secretion system. In some embodiments, the bacteria are engineered to programmably express a virulence gene such as genetic components of other bacterial surface markers (e.g., LPS, fimbrae, pili) in response to external modulators.

In some embodiments, the programmable bacterial cells belong to at least one genus selected from the group consisting of Salmonella, Escherichia, Firmicutes, Bacteroidetes, Lactobacillus, and Bifidobacteria. In some embodiments, the bacterial cells belong to more than one genus selected from the group consisting of Salmonella, Escherichia, Firmicutes, Bacteroidetes, Lactobacillus, and Bifidobacteria.

In some embodiments, the programmable bacterial cells belong to the genus Escherichia. In particular embodiments, the programmable bacterial cells are Escherichia coli Nissle 1917 (EcN) cells. In some embodiments, the programmable bacterial cells comprise a gene that regulates capsular polysaccharide nanoencapsulation selected from the group consisting of kfi and kps genes. In some embodiments, the programmable bacterial cells comprise a gene that regulates capsular polysaccharide nanoencapsulation selected from the group consisting of kfiA, kfiB, kfiC, kfiD, kpsE, kpsD, kpsM, kpsT, kpsC, kpsS, kpsF and kpsU.

Some aspects of this invention implicitly relate to culturing the programmable bacterial cells described herein. In some embodiments, a culture comprises the programmable bacterial cells and a medium, for example, a liquid medium, which may also comprise: a carbon source, for example, a carbohydrate source, or an organic acid or salt thereof; a buffer establishing conditions of salinity, osmolarity, and pH, that are amenable to survival and growth; additives such as amino acids, albumin, growth factors, enzyme inhibitors (for example protease inhibitors), fatty acids, lipids, hormones (e.g., dexamethasone and gibberellic acid), trace elements, inorganic compounds (e.g., reducing agents, such as manganese), redox-regulators (e.g., antioxidants), stabilizing agents (e.g., dimethyl sulfoxide), polyethylene glycol, polyvinylpyrrolidone (PVP), gelatin, antibiotics (e.g., Brefeldin A), salts (e.g., NaCl), chelating agents (e.g., EDTA, EGTA), and enzymes (e.g., cellulase, dispase, hyaluronidase, or DNase). In some embodiments, the culture may comprise an agent that induces or inhibits transcription of one or more genes in operable linkage with an inducible promoter, for example doxicycline, tetracycline, tamoxifen, IPTG, hormones, or metal ions. While the specific culture conditions depend upon the particular programmable bacterial cells, general methods and culture conditions for the generation of microbial cultures are well known to those of skill in the art.

### Therapeutic Methods and Compositions

The inventions described herein also encompass methods of treating a tumor in a subject comprising administering a therapeutically effective amount of programmable bacterial cells described herein to the subject, wherein the programmable bacterial cells comprise a nucleic acid encoding a therapeutic agent described herein, which capable of treating the tumor. The present disclosure also relates to methods of reducing the rate of proliferation of a tumor cell comprising delivering a programmable bacterial cell described herein to the tumor cell. The present disclosure also relates to methods of killing a tumor cell comprising delivering a programmable bacterial cell described herein to the tumor cell. In some embodiments, the tumor or tumor cell is from a colorectal tumor. In some embodiments, the tumor cell is from a breast cancer.

As used interchangeably herein, “treatment” or “treating” or “treat” refers to all processes wherein there may be a slowing, interrupting, arresting, controlling, stopping, alleviating, or ameliorating symptoms or complications, or reversing of the progression of cancer, but does not necessarily indicate a total elimination of all disease or all symptoms. Non-limiting examples of treatment include reducing the rate of growth of a tumor, reducing the size of a tumor, or preventing the metastases of a tumor.

Programmable bacterial cells described herein are preferably administered in one or more therapeutically effective doses. As used herein the terms “therapeutically effective dose” means the number of cells per dose administered to a subject in need thereof that is sufficient to treat the hyperproliferative disorder. In some embodiments, a therapeutically effective dose can be at least about 1×10⁴ cells, at least about 1×10⁵ cells, at least about 1×10⁶ cells, at least about 1×10⁷ cells, at least about 1×10⁸ cells, at least about 1×10⁹ cells, or at least about 1×10¹⁰ cells.

In some embodiments, programmable bacterial cells may be delivered to a subject in the form of a pharmaceutical composition, which may comprise one or more pharmaceutically acceptable carriers, diluents, or excipients. Pharmaceutical compositions may be formulated as desired using art recognized techniques. Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents, and the like, are also available. Certain non-limiting exemplary carriers include saline, buffered saline, dextrose, water, glycerol, ethanol, and combinations thereof. Pharmaceutical compositions may be frozen and thawed prior to administration or may be reconstituted in WFI with or without additional additives (e.g., albumin, dimethyl sulfoxide). Programmable bacterial cells described herein are preferably formulated for oral, intravenous, subcutaneous, or intratumoral administration, but other routes of administration known in the art may be utilized.

Particular dosage regimens, i.e., dose, timing, and repetition, will depend on the particular subject being treated and that subject's medical history. Empirical considerations such as pharmacokinetics will contribute to the determination of the dosage. Frequency of administration may be determined and adjusted over the course of therapy and is based on reducing the number of tumor cells or tumor mass, maintaining the reduction of such tumor cells or tumor mass, reducing the proliferation of tumor cells or an increase in tumor mass, or delaying the development of metastasis. A therapeutically effective dose may depend on the mass of the subject being treated, his or her physical condition, the extensiveness of the condition to be treated, and the age of the subject being treated.

### Articles of Manufacture

The inventions disclosed herein also encompass articles of manufacture useful for treating a colorectal tumor comprising a container comprising programmable bacterial cells described herein, or a pharmaceutical composition comprising the same, as well as instructional materials for using the same to treat the colorectal tumor. In some embodiments, the articles of manufacture are part of a kit that comprises a bacterial culture vessel and/or bacterial cell growth media.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Bacterial Strains and Culturing

The host strain used in this study was Escherichia coli Nissle 1917 (EcN) that naturally expresses K5 capsular polysaccharide (CAP) containing a genomically integrated erythromycin-resistance luxCDABE cassette for bacterial bioluminescence tracking in vivo. All bacteria were grown with appropriate antibiotics selection (100 μg/mL ampicillin, 50 μg/mL kanamycin, 25 g/mL chloramphenicol, 50 μg/mL erythromycin) in LB media (Sigma-Aldrich) at 225 RPM or on LB-agar plates containing 1.5% agar at 37° C.

### Example 2

### Construction of Plasmids and Gene Circuits

To construct a knockdown library, plasmids with sRNA targeting each gene of the CAP biosynthetic pathway were prepared using Gibson Assembly. The sRNA sequences were designed to be complementary and bind to the 24-neucleotide sequence of the target gene coding sequence spanning the ribosome binding site and the start codon. A plasmid template was prepared by PCR-amplifying backbone (pTH05) using primers (pTH05_for and pTH05 rev), and the single-stranded DNA for sRNA against genes in CAP biosynthesis were inserted (kfiA, kfiB, kfiD, kpsC, kpsS, kpsF, kpsU, kpsE, kpsD, kpsT, kpsM), and transformed into MACH1™ competent cells (Invitrogen). CAP gene circuits and the therapeutic plasmids were constructed in a similar manner. Genes of interest were obtained by synthesizing oligos or GBLOCK™ from IDT, or PCR-amplification (kfiC gene was obtained via colony PCR from EcN). Subsequently, plasmids were constructed using Gibson Assembly or using standard restriction digest and ligation cloning, and transformed into MACH1™ competent cells (Invitrogen).

### Example 3

### Construction of Knockout Strains

EcN was transformed to carry Lambda Red helper plasmid (pKD46). Transformants were grown in 50 mL LB at 30° C. with chloramphenicol to an OD₆₀₀ of 0.4 and made electrocompetent by washing three times with ice cold MilliQ water and concentrating 150-fold in 15% glycerol. Chloramphenicol-resistance cassette was prepared by PCR with primers flanked by sequence within each target gene followed by gel purification and resuspension in MilliQ water Electroporation was performed using 50 μL of competent cells and 10-100 ng of DNA. Shocked cells were added to 1 mL SOC, incubated at 30° C. for 1 hour with 20 μL arabinose, and incubated at 37° C. for 1 hour. Cells were then plated on LB plates with chloramphenicol and incubated in 37° C. overnight. Colonies were picked the next day to obtain knockout strains including ΔkfiC strain (EcN ΔkfiC).

### Example 4

### Characterization of CAP Strains Sensitivity to Phages, Antibiotics, and Acids

To perform plaque forming assay, bacteria were plated onto LB agar plates to make a lawn and allowed to dry under fire. 10 μL of serial diluted ΦK1-5 phage (Molineux, University of Texas, Austin) was spotted onto the plates and allowed to dry. Plates were incubated at 37° C. overnight and inspected the next day for plaque forming unit (PFU) counting. Similar phage plaque forming assay were performed for K1 and K5 type E. coli strains.

To assess bacterial growth in liquid culture, overnight cultures of EcN, EcN ΔkfiC, or EcN iCAP strains were calibrated into OD₆₀₀ of 1.0, and 100 μL of each was transferred into 96-well plate (Corning). 1 μL of 10⁸ PFU ΦK1-5 phage, or antibiotics of indicated concentrations were added to each well. The samples were incubated at 37° C. with shaking in Tecan plate reader, and the OD₆₀₀ was measured every 20 min. For bacterial growth in low pH condition, LB media adjusted to pH 2.5 using HCl, bacteria were incubated at 37° C. for 1 hour, followed by serial dilution and plating on a LB agar plate for CFU enumeration.

### Example 5

### Characterization of CAP Using SDS-PAGE

CAP was purified via the chloroform-phenol extraction as previously described. Briefly, 3 mL of overnight bacteria cultures were harvested the next day and further sub-cultured in 50 mL LB broth in the presence or absence of 0.1 M IPTG for indicated lengths of time. Bacteria concentrations were adjusted to the same level across samples via OD₆₀₀ before centrifugation. Pellets were collected and resuspended in 150 μL of water. An equal amount of hot phenol (65° C.) was added, and the mixtures were vortexed vigorously. The mixtures were then incubated at 65° C. for 20 minutes, followed by chloroform extraction (400 μL) and centrifugation. The CAP were detected by Alcian blue staining as previously reported. Briefly, following SDS-PAGE electrophoresis (4-20% gradient), the gel was fixed in fixing solution (25% ethanol, 10% acetic acid in water) for 15 minutes while shaking at room temperature. The gel was then incubated in Alcian blue solution (0.125% Alcian blue in 25% ethanol, 10% acetic acid in water) at room temperature for 2 hours while shaking before de-stained overnight in fixing solution. CAP was visualized as Alcian blue stained bands on the resulting gel.

### Example 6

### Visualization of CAP Using TEM

Bacteria were grown overnight in LB media with appropriate antibiotics before being processed for imaging. For EcN iCAP, a 1:100 dilution in LB with antibiotics was made the following day and grown in 37° C. shaker until OD₆₀₀=0.1-0.4 (mid-log phase), and varying concentrations of IPTG were added for further incubation for 6 hours before being processed. The cultures were spun down at 300 relative centrifugal force (rcf) for 10 min and embedded in 2% agarose. Each agarose gel fragment was cut into a cube with 2-mm edge and placed in a 1.5-mL centrifuge tube. The samples embedded in agarose were fixed and stained via protocols previously reported. Briefly, the samples were fixed with 2% paraformaldehyde and 2.5% glutaraldehyde in osmotically adjusted buffer (0.1 M sodium cacodylate, 0.9 M sucrose, 10 mM CaCl₂, 10 mM MgCl₂) with 0.075% ruthenium red and 75 mM lysine acetate for 20 min on ice. The samples were washed with osmotically adjusted buffer containing 0.075% ruthenium red twice and further fixed with 1% osmium tetroxide in osmotically adjusted buffer containing 0.075% ruthenium red for an hour on ice. The samples were washed three times in water with 5 min incubation between each wash and dehydrated in increasing concentrations of ethanol (50%, 70%, and 100%) on ice for 15 min per step. The samples were washed one more time in 100% ethanol and embedded in increasing concentrations of Spurr's resin (33% and 66%) diluted in ethanol for 30 min per step and overnight in 100% Spurr's resin. The samples were moved to fresh Spurr's resin the next day and polymerized at 65° C. overnight before sectioned using SORVALL MT-2B Ultramicrotome to ˜70 nm. The sample sections were placed on TEM grids (Ted Pella; 01800F) and stained using UranyLess (EMS). The sample grids were imaged using FEI Talos 200 TEM.

### Example 7

### TEM Image Processing and Data Analysis of Polysaccharide Layer

The image processing of TEM images was performed using ImageJ, and the data analysis was done using MATLAB. Due to low signal-to-noise ratio of the TEM images resulting from thinly sectioned bacteria samples stained using ruthenium red, Gaussian blur was used to reduce the noise and help determine the boundary of the polysaccharide layer. Polysaccharide layer was selected and transformed into binary image using threshold function. Some portion of boundary of polysaccharide layer was manually outlined when the thresholding function could not determine where the boundary was. The resulting binary image of polysaccharide layer was used to identify the centroid and measure distribution of polysaccharide thickness in respect to the centroid. For each sample, five representative images were used to measure the polysaccharide thickness and the measurements were aggregated to form histograms. The resulting histograms were fitted with Gaussian curves to extract mean and standard deviation of polysaccharide layer thickness.

### Example 8

### In Vitro Whole Blood Bactericidal Assays

EcN, EcN ΔkfiC, or EcN iCAP bacterial cultures were grown overnight in LB broth with appropriate antibiotics and IPTG concentrations. The cultures were spun down at 3000 rcf for 5 min and resuspended in 1 mL sterile PBS. They were further normalized to an OD₆₀₀ of 1 with sterile PBS. 150 μL of blood from the single donor human whole blood or murine (BALB/c) whole blood (Innovative Research) were aliquoted into 3 wells/strain in a 96-well plate. 1.5 μL of bacteria were added to each well and incubated at 37° C. At various time points, the plate was taken out, and a serial dilution of each sample was prepared in PBS. The dilutions were plated on LB agar plates with erythromycin. The agar plates were incubated at 37° C. overnight and inspected the next day for CFU counting.

### Example 9

### Phagocytosis Assays

The phagocytosis assays were performed via protocols as previously reported 9, 10. Briefly, bone marrow derived macrophages (BMDM) were thawed on a 15 cm non-TC treated petri dish and cultured in RPMI with 10% FBS and MCSF for 4 days before experiment. On the 4th day, BMDMs were collected, counted, and diluted to 2×10⁵ cells/mL in RPMI with 10% FBS (without antibiotics). Afterwards, 1 mL of the new mixture was plated per well (2×10⁵ cells) in a 24-well TC-treated plate and cultured overnight. Media in the 24-well BMDM plate was removed the next day, and 1 mL of EcN iCAP constitutively expressing GFP with or without IPTG induction were resuspended in RPMI with 10% FBS without antibiotics was added into each well at MOI of 100. The co-culture was incubated for 30 min at 37° C. followed by rigorous washing with PBS at least 3 times. 1 mL of RPMI with 10% FBS and gentamicin (30 μg/mL) was added to each well, followed by live imaging under confocal microscopy. 0.1 M IPTG was added to EcN iCAP with IPTG induction the entire time. Then, the BMDMs were lysed with 0.5% TRITON X-100® in PBS and lysates were collected and plated on LB agar with erythromycin followed by overnight incubation at 37° C. Colonies were counted the next day. ImageJ was used to count the number of macrophages, engulfed bacterial cells, and macrophages containing engulfed bacterial cells from the confocal images. The phagocytic index was calculated according to the following formula: phagocytic index=(total number of engulfed bacterial cells/total number of counted macrophages)×(number of macrophages containing engulfed bacterial cells/total number of counted macrophages)×100.

### Example 10

### Determination of TNFα Response

THP-1 cells (ATCC) were maintained in RPMI-1640 supplemented with 10% FBS, 2 mM L-glutamine, 100 μg/mL streptomycin, 100 μg/mL penicillin, and 0.1% mercaptoethanol at 37° C. and 5% CO₂. Cells were passaged every 72 hours. For cell quantification and viability analysis, cells were stained using trypan blue stain. For in vitro TNFα assay, THP-1 was resuspended at a concentration of 1×10⁶ cells/mL in RPMI-1640 supplemented with 10% FBS and 0.1% gentamycin. 300 μL of cell suspension was transferred into each well of a 24-well plate. 3 μL of each bacterial strain at each concentration were added to cell culture wells.

Subsequently, the culture medium was harvested and centrifuged at 200 rcf for 5 min to isolate THP-1 without causing cell death. Supernatant was then centrifuged at 3000 rcf for 5 min to remove bacteria. The resulting supernatant was analyzed for TNFα response. TNFα was measured using an R&D Systems Quantikine ELISA Kit in a plate reader.

### Example 11

### Animal Models

All animal experiments were approved by the Institutional Animal Care and Use Committee (Columbia University, protocols ACAAAN8002 and AC-AAAZ4470). For tumor-bearing animals, euthanasia was required when the tumor burden reaches 2 cm in diameter or after recommendation by the veterinary staff. Mice were blindly randomized into various groups.

Animal experiments were performed on 8-12 weeks-old female BALB/c mice (Taconic Biosciences). Tumor models were established with bilateral subcutaneous hind flank injection of mouse colorectal carcinoma CT26 cells (ATCC) or mammary fat pad injection of 4T1-luciferase mammary carcinoma cells (Kang, Princeton University). The concentration for implantation of the tumor cells was 5×10⁷ cells per ml in RPMI (no phenol red). Cells were injected at a volume of 100 μL per flank, with each implant consisting of 5×10⁶ cells. Female transgenic MMTV-PyMT mice (Jackson Laboratory) which develops mammary tumors were also used. Tumors were grown to an average of approximately 200-400 mm³ before experiments. Tumor volume was quantified using calipers to measure the length, width, and height of each tumor (V=L×W×H). Because the z dimension of PyMT tumor is highly variable, total volume was calculated as length×width²×0.5. Volumes were normalized to pre-injection values to calculate relative or % tumor growth on a per mouse basis.

### Example 12

### Bacterial Administration for In Vivo Experiments

Overnight cultures of EcN, EcN ΔkfiC, and EcN iCAP were grown in LB medium with the appropriate antibiotics and inducers. A 1:100 dilution in LB with appropriate antibiotics and inducers was made the following day and grown in 37° C. shaker until OD₆₀₀=0.1-0.4 (mid-log phase). Cultures were centrifuged at 3000 rcf for 10 min and washed three times with cold sterile PBS. The bacteria were then normalized to a desired OD₆₀₀. Unless otherwise noted, intravenous injections were given through the tail-vein at the dose of 5×10⁶ cells/mL (OD₆₀₀ of 0.5) in PBS with a total volume of 100 μL per mouse. Intratumoral injections of bacteria were performed at a concentration of 5×10⁶ cells/mL with a total volume of 40 μL per tumor. Intraperitoneal injections were injected at varying concentrations in PBS with a total volume of 100 μL per mouse. For induction of theta toxin production, AHL subcutaneous injection was given to mice daily at 10 μM concentration with a total volume of 500 μL per mouse. For in situ activation of iCAP, water containing 10 mM IPTG was given to mice a day after bacterial administration.

### Example 13

### Biodistribution and In Vivo Animal Imaging

All bacterial strains used in this study had integrated luxCDABE cassette that could be visualized by IVIS spectrum imaging system (Perkin Elmer) and were quantified by Living Image software.

Images and body weight of mouse were obtained every day starting the day of bacterial administration until the study endpoint. At the study endpoint, mice were euthanatized by carbon dioxide, and the tumors and organs (spleen, liver, and lungs) were extracted and imaged. They were later weighed and homogenized using a GENTLEMACS® tissue dissociator (C Tubes, Miltenyi Biotec). Homogenates were serially diluted with sterile PBS and plated on LB agar plates with erythromycin and incubated overnight at 37° C. Colonies were counted the next day.

### Example 14

### Statistical Analysis

Statistical tests were performed either in GraphPad Prism 7.0 (Student's t-test and ANOVA) or Microsoft Excel. The details of the statistical tests are indicated in the respective figure legends. When data were approximately normally distributed, values were compared using either a Student's t-test, one-way ANOVA for single variable, or a two-way ANOVA for two variables. Mice were randomized into different groups before experiments.

### Example 15

sRNA Knockdown Screen Identifies Key Regulators of CAP Synthesis

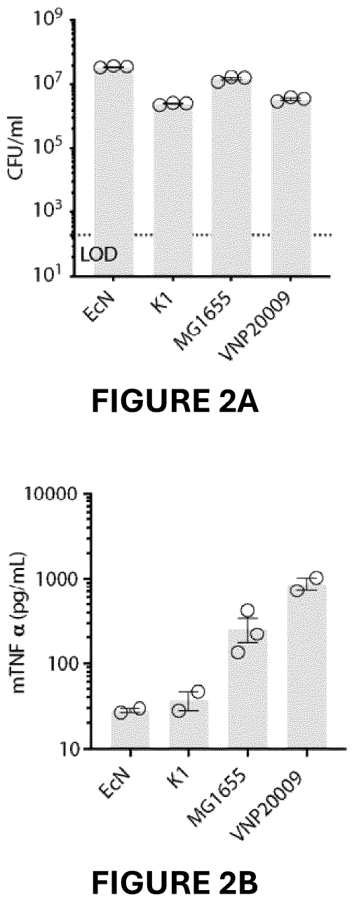

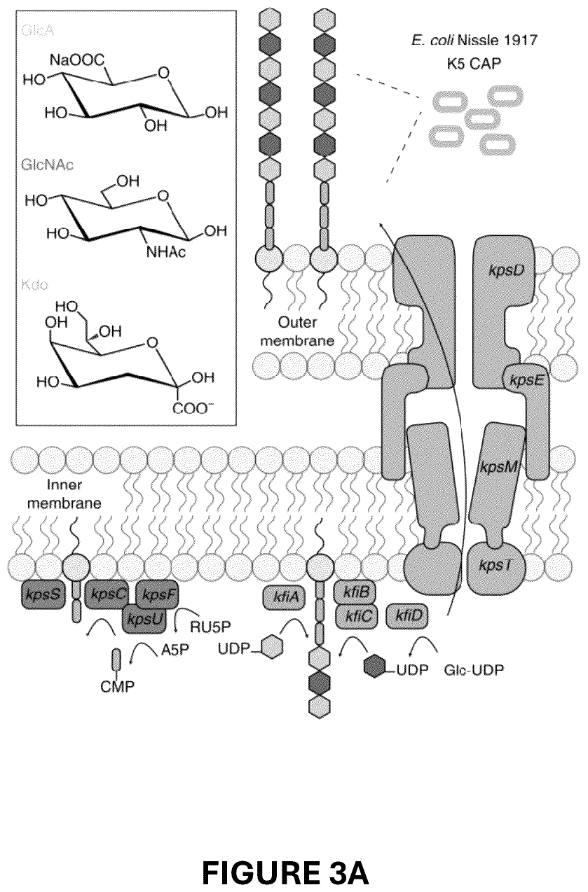

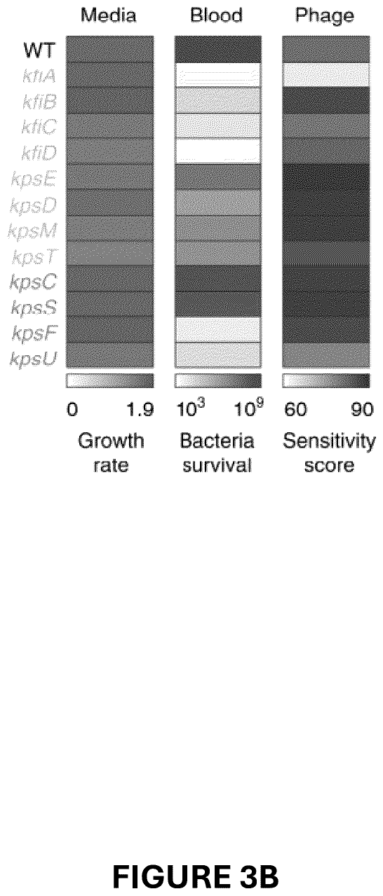

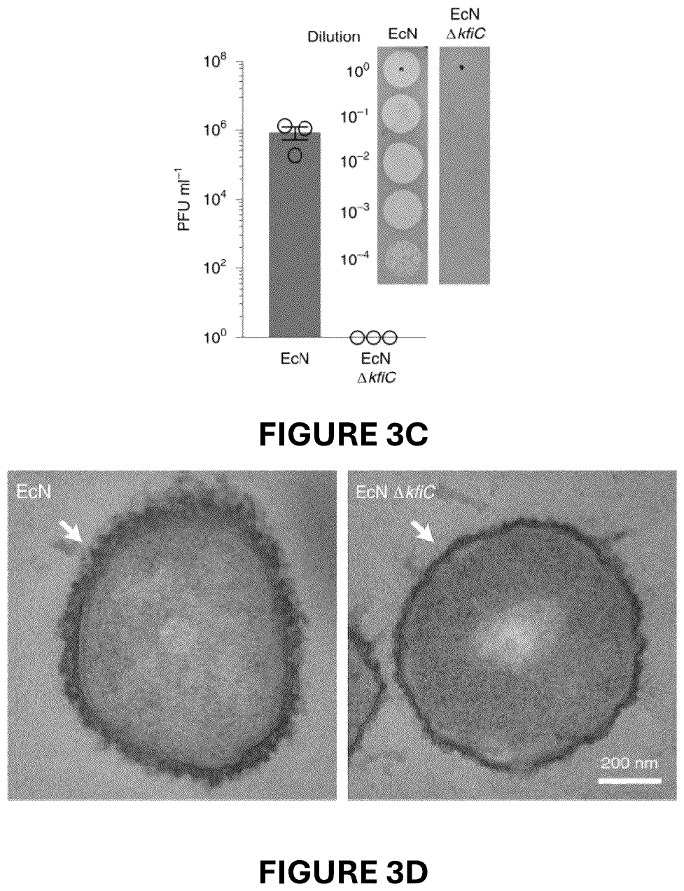

Since various bacteria have been utilized for therapeutic applications, the immunogenicity and viability of several E. coli and S. typhimurium strains were compared. Here E. coli Nissle 1917 (EcN), a probiotic strain with favorable clinical profiles, demonstrated high viability in human whole blood with minimal cytokine induction (FIGS. 2a and b). Because the K5-type CAP of EcN has been shown to alter interaction with host immune systems, we chose to genetically modify its biosynthetic pathway. K5-type CAP produced from EcN, also known as heparosan, is composed of a polymer chain of alternating β-D-glucuronic acid (GlcA) and N-acetyl-α-D-glucosamine (GlcNAc), attached to β-deoxy-D-manno-oct-2-ulosonic acid (Kdo) linker (FIG. 3a). Glycotransferases of kfABCD genes polymerize alternating GlcA and GlcNAc subunits. kpsCSFU genes are responsible for synthesis of the poly-Kdo linker on the terminal lipid, and CAP is transported to the cellular surface by kpsEDMT genes. While individual functions of the CAP genes have been investigated, engineering tunable and dynamic control of this system remains unexplored.

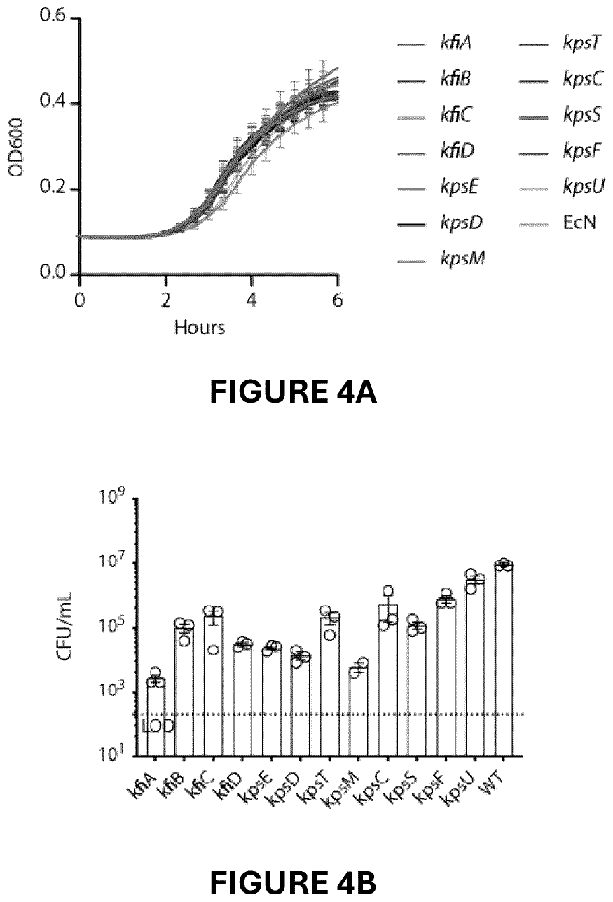

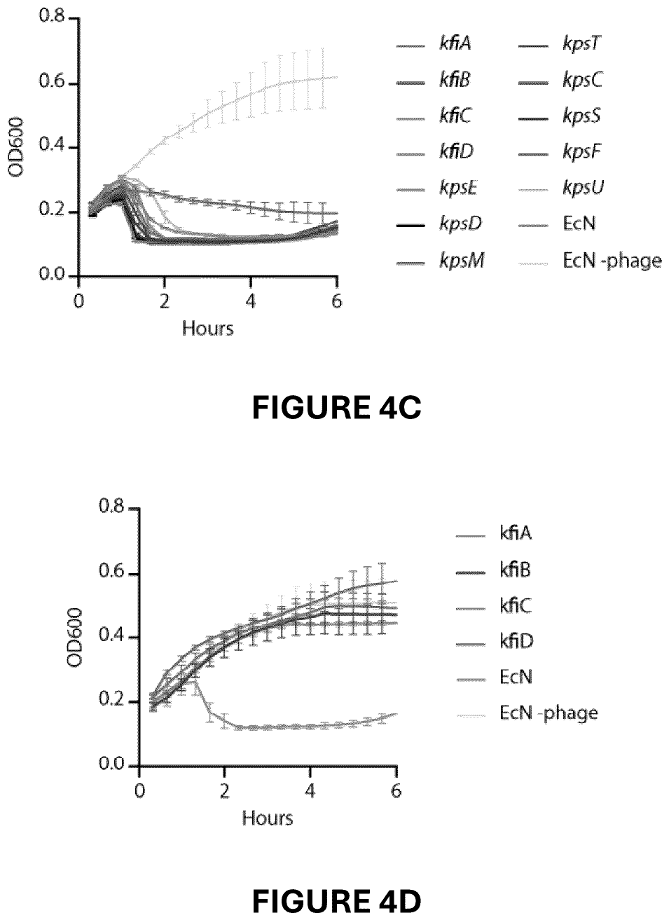

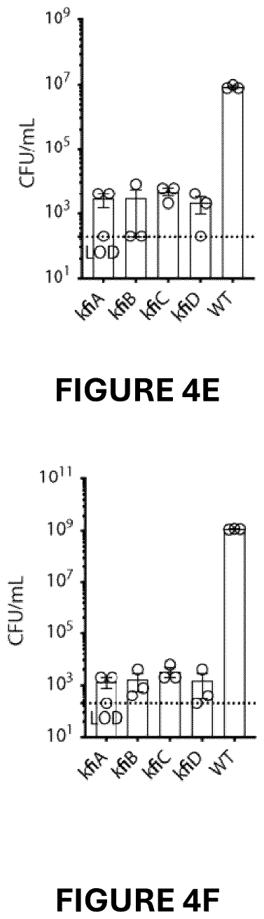

To identify key CAP genes capable of altering response to antibacterial factors encountered during therapeutic delivery, a library of knockdown (KD) strains using synthetic small RNAs (sRNAs) that reduce expression of kfi and kps genes via complementary binding to mRNAs was generated. To initially assess the impact of downregulating each gene, the growth of KD strains in (1) nutrient-rich media, (2) human whole blood, and (3) CAP-targeting phage was assessed. Growth in nutrient-rich media showed little variation in maximum specific growth rates (μm) from the wild-type EcN strain (expressing CAP) (FIGS. 3b and 4a), suggesting that the downregulation of the targeted genes in the CAP biosynthetic pathway does not greatly affect the fitness of EcN in the absence of environmental threats.

Significantly reduced viability of KD strains compared to EcN was noted after incubation in whole blood for 0.5 hours (FIG. 4b). After a 6-hour incubation in whole blood, KD strains in CAP synthesis (kfi genes and kpsFU) exhibited lower viability compared to KD strains in CAP transport (kpsEDMT) (FIG. 2b). To assess whether each gene KD caused a complete or partial loss of CAP in KD strains, lytic bacteriophage ΦK1-5 that specifically binds to heparosan of EcN were used. Bacteria that express residual levels of heparosan CAP are susceptible to this phage, but complete loss of CAP confers immunity. Phage sensitivity of each strain was quantified by measuring the area under the curve of the phage-inoculated growth curve and observed that sRNA KD of most CAP genes did not alter phage sensitivity compared to control EcN (FIGS. 3b and 4c), suggesting some level of CAP was still present in KD strains.

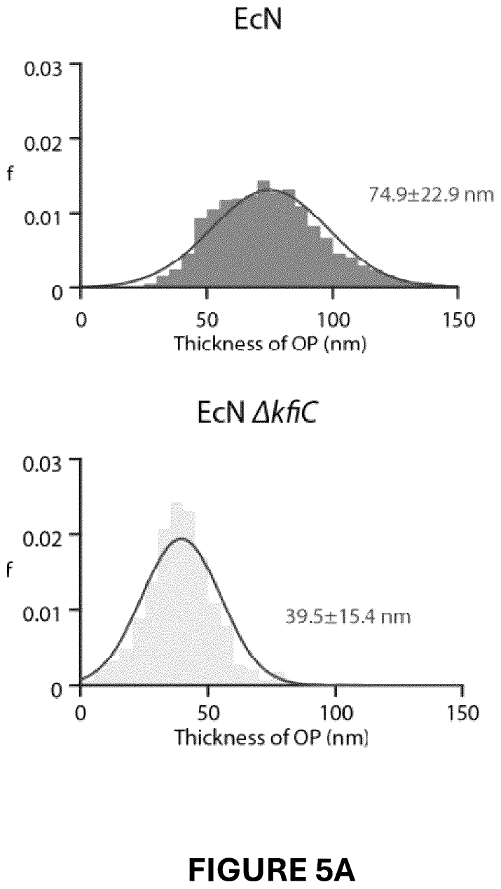

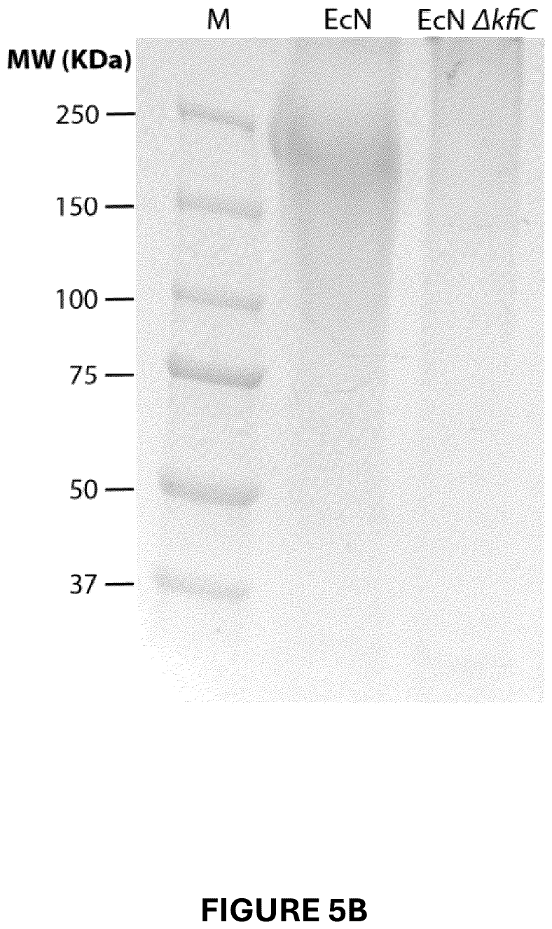

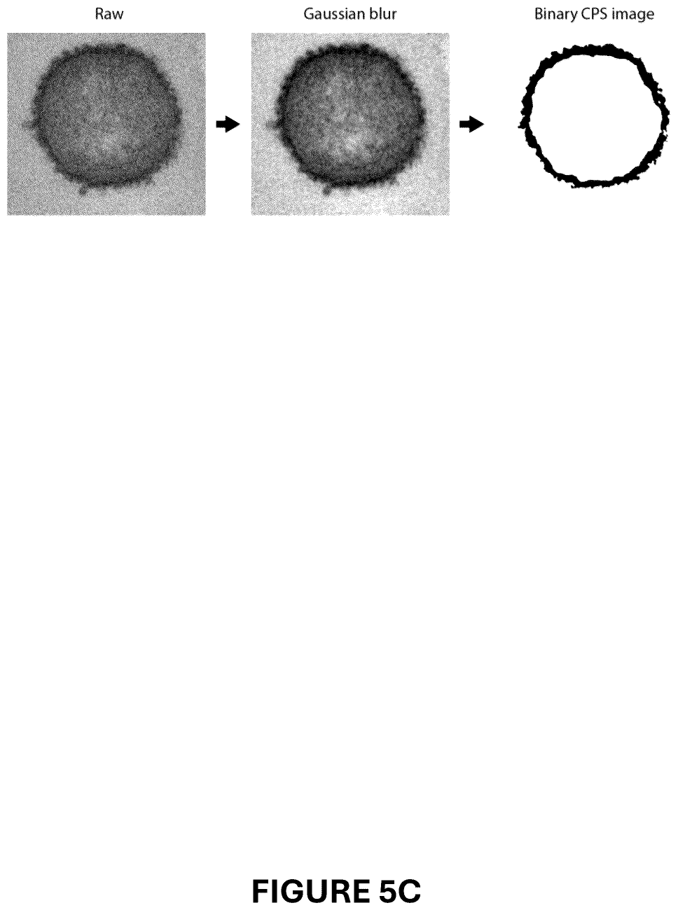

To abrogate the effect of residual CAP gene expression, a library of knockout (KO) strains was constructed by deleting CAP synthesis genes from EcN genome using Lambda Red recombineering system. All kfi KO strains resulted in complete phage immunity (FIG. 5d), supporting loss of CAP expression. Importantly, KO strains demonstrated significantly increased blood sensitivity (FIG. 4e), indicating that CAP levels can be genetically tuned to alter sensitivities to antibacterial factors.

On the basis of these results, kfiC, a well-studied gene that encodes an essential glycotransferase of GlcA, was further characterized. Downregulation of kfiC via sRNA KD sensitized bacteria in blood, suggesting its key role in regulating bacterial protection. Deletion of kfiC resulted in the highest enhancement in blood sensitivity, indicating that the level of protection can be altered by controlling gene expression. To confirm loss of CAP from the bacterial surface, the surface properties of EcN ΔkfiC strain were ascertained. Phage plaque formation assay confirmed complete immunity against ΦK1-5 (FIG. 3c). Bacterial polysaccharides were also detected using sodium dodecyl sulfate-polyacrylamide gel electrophoresis (SDS-PAGE) followed by CAP staining with Alcian blue. Compared to EcN that produced strong staining with Alcian blue at ˜180 kDa, EcN ΔkfiC produced no visible band (FIG. 5a).

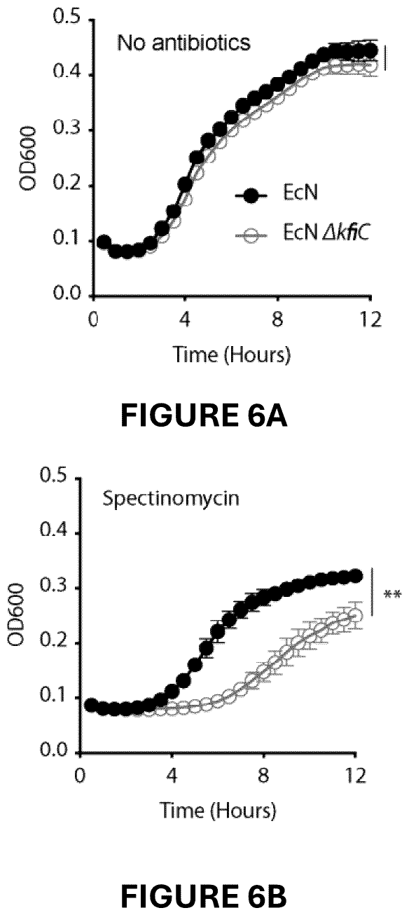

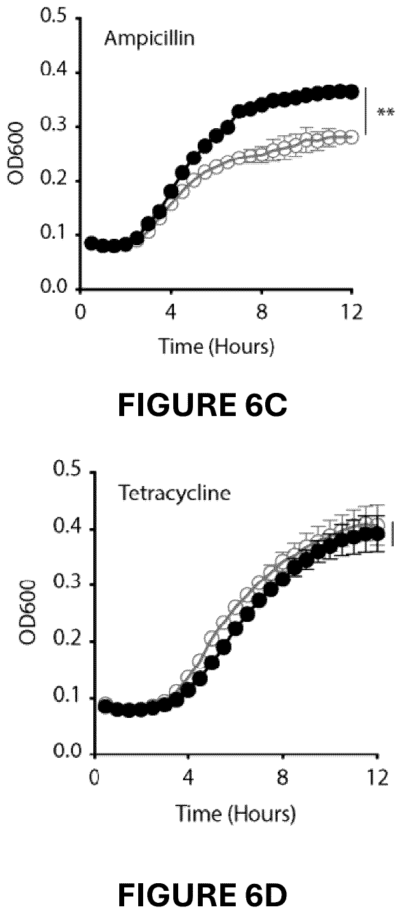

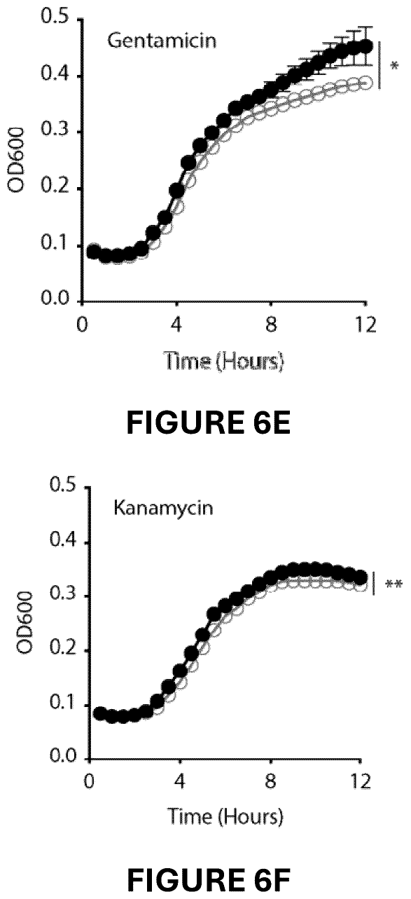

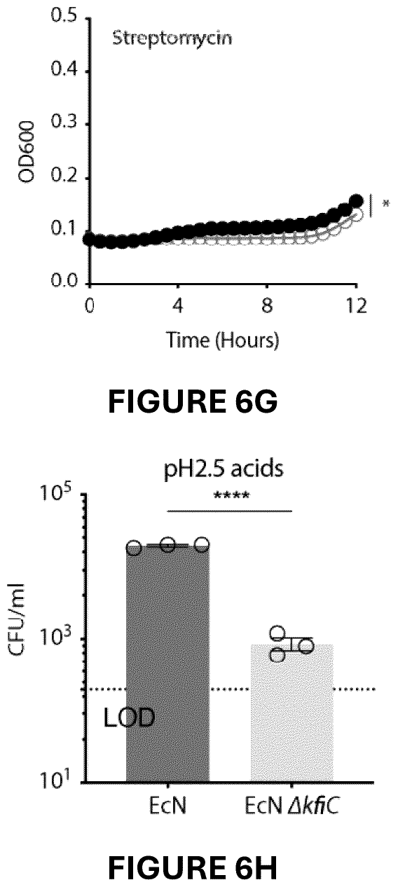

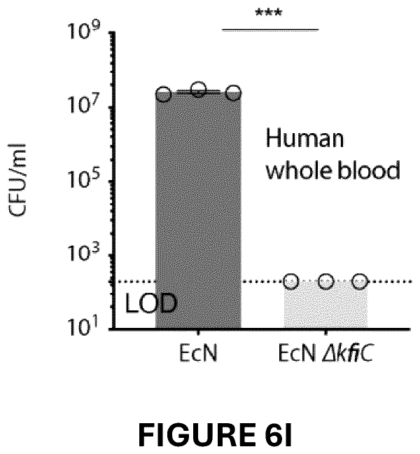

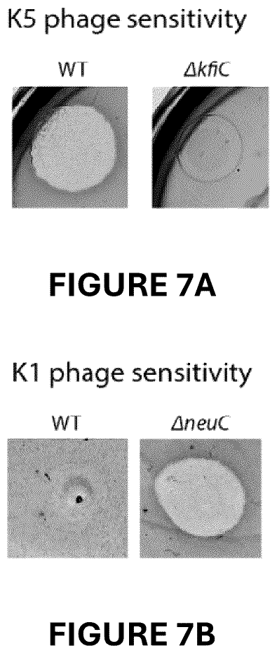

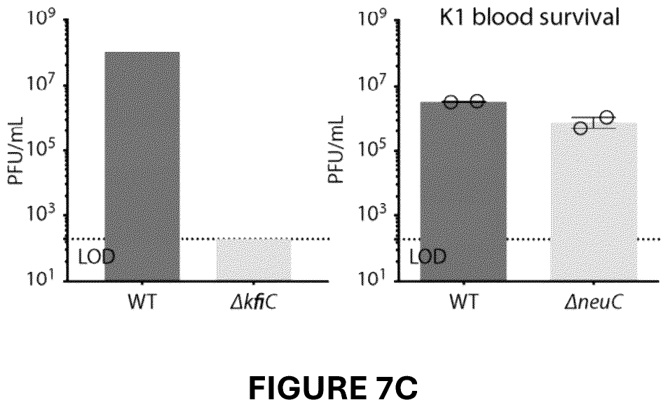

The morphological changes in bacterial surface were also characterized using transmission electron microscopy (TEM) with ruthenium red staining. CAP was visible as an ˜80 nm thick layer of polysaccharides coating outside of the cellular membrane. In contrast, EcN ΔkfiC had diminished size of the polysaccharides layer at ˜40 nm (FIGS. 3d and 5a and b). The capability of CAP to protect cells from wide range of antimicrobial factors was also investigated. In addition to the modified sensitivity to human whole blood and bacteriophage, EcN ΔkfiC demonstrated a significant reduction in cellular protection against panels of antibiotics (spectinomycin, ampicillin, gentamicin, kanamycin, streptomycin) and extreme acids (pH 2.5) compared to EcN (FIG. 6a-i). Finally, the general applicability of the approach in other CAP systems was evaluated. Homologous genes in different E. coli strains expressing K1 and K5 CAP (neuC and kfiC, respectively) were deleted and alteration in environmental sensitivity was observed (FIG. 7a-c). Together, these results demonstrate that loss of CAP modifies cellular surface structure and protection against antimicrobial factors.

### Example 16

### Construction of Tunable and Reversible Programmable CAP

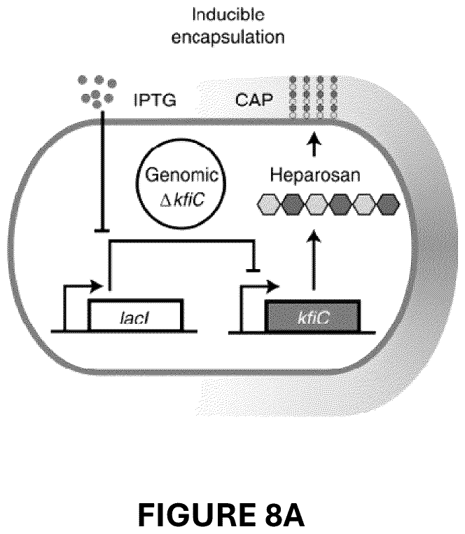

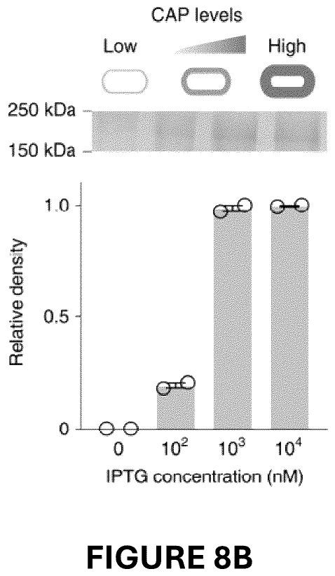

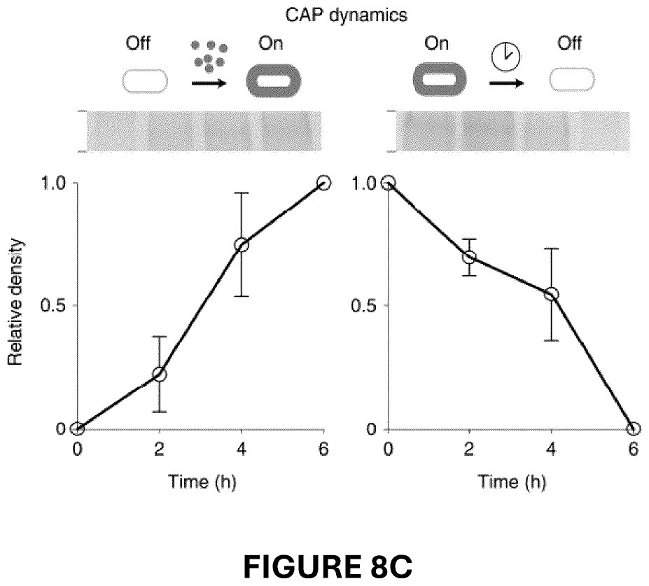

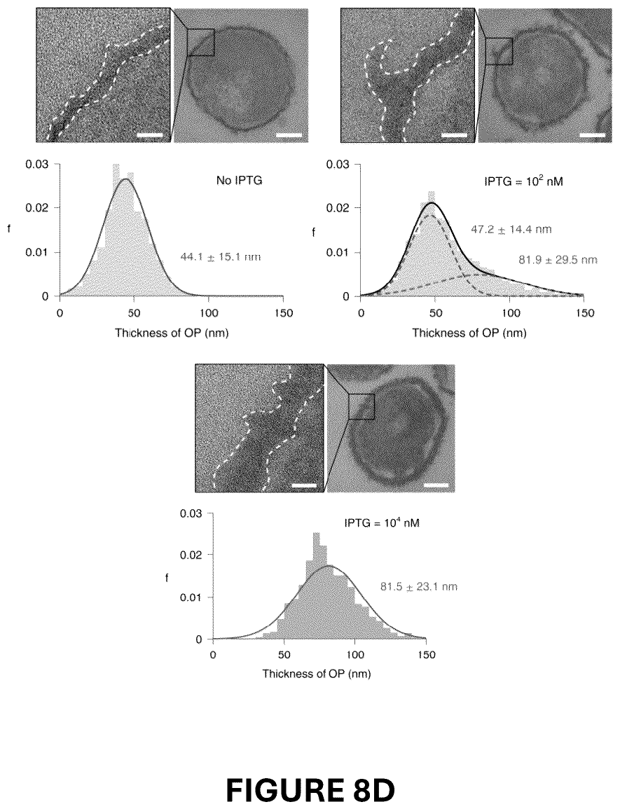

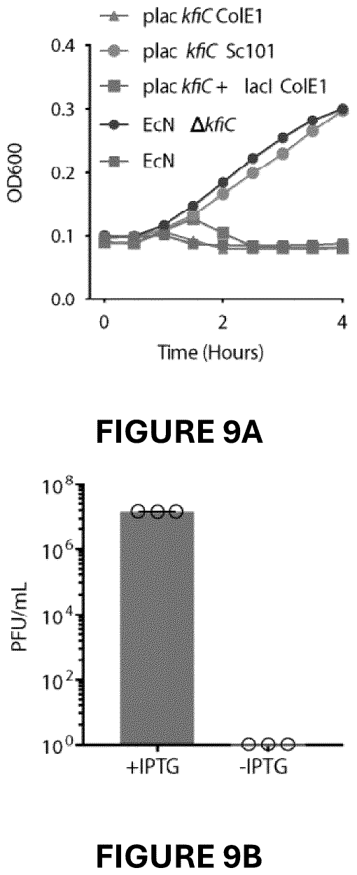

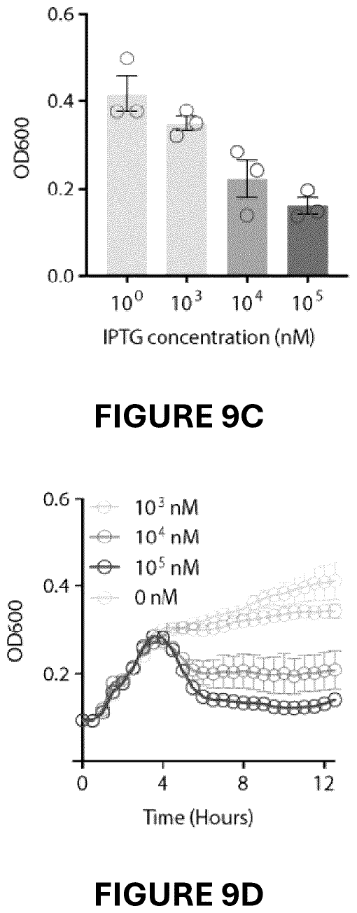

A programmable CAP system that can sense and respond to induction stimuli and modulate cell surface properties was created by putting kfiC under the control of the tac promoter, which can be activated with the small-molecule inducer isopropyl-b-D-thiogalactopyranoside (IPTG) (FIG. 8a). A small library of plasmids with various copy numbers of kfiC was created to optimize for tight regulation of CAP production. EcN ΔkfiC transformed with the low (sc101 origin) copy number plasmid exhibited complete immunity against ΦK1-5 (FIG. 9a), indicating tight repression at the basal level. Induction with IPTG rescued the phage sensitivity (FIG. 9b), confirming inducible modulation of CAP on cellular surface. Tunability of this inducible CAP (iCAP) system was examined by characterizing multiple induction conditions. SDS-PAGE showed increase in CAP production from EcN carrying the iCAP system (EcN iCAP) when incubated with elevating levels of IPTG (FIG. 8b). Co-incubation with ΦK1-5 also showed decreasing viability of EcN iCAP with elevating levels of IPTG (FIG. 9c). TEM was subsequently used to investigate the effect of the iCAP system on cell surface morphology (FIG. 8d). Increasing levels of IPTG shifted the mean bacterial membrane thickness from 44 nm to 81 nm, confirming tunable capability of the system. Intermediate iCAP activation at 100 nM revealed a bimodal distribution of the membrane thickness, suggesting that kfiC regulates the production level but not the length of the polysaccharide polymers. This result agreed with SDS-PAGE data that showed no difference in migration of CAP band depending on IPTG concentration (FIG. 8b).

The dynamics of production and recovery of the iCAP system were subsequently evaluated using a similar approach. Upon addition of IPTG, elevated CAP production was observed over time on SDS-PAGE, reaching near-maximum levels by 4 hours (FIG. 8c). Similarly, removal of IPTG resulted in gradual decrease in CAP until complete repression by 6 hours. iCAP dynamics were also tested via co-incubation with ΦK1-5. While uninduced EcN iCAP grew, induction with IPTG at the start of co-incubation resulted in a rapid lysis event at 3.5 hours (FIG. 9d), demonstrating delayed CAP production with similar kinetics observed in SDS-PAGE. Collectively, these data highlight the programmable capability of CAP modulation on the bacterial surface.

### Example 17

Programmable Protection from Host Immunity

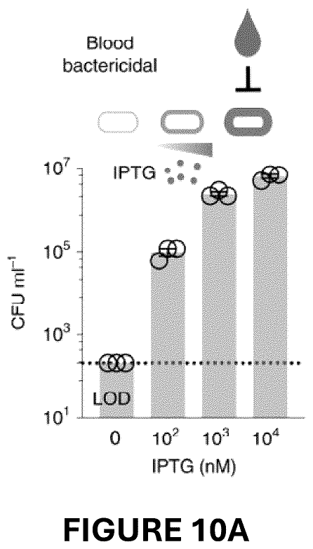

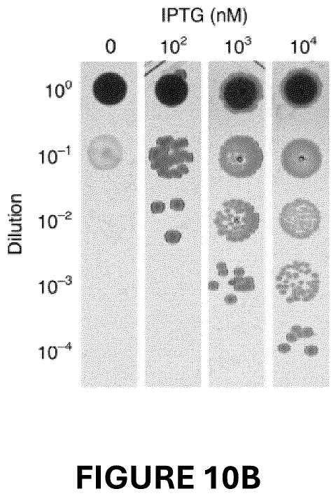

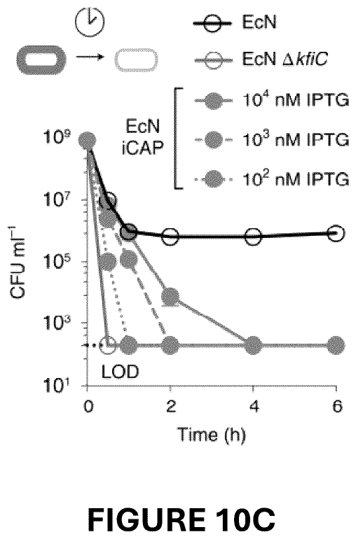

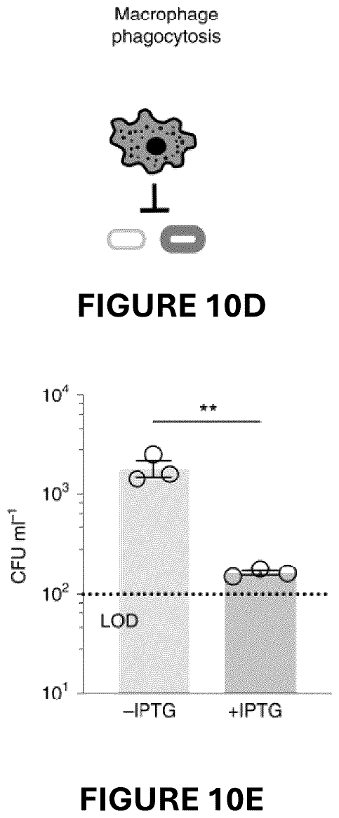

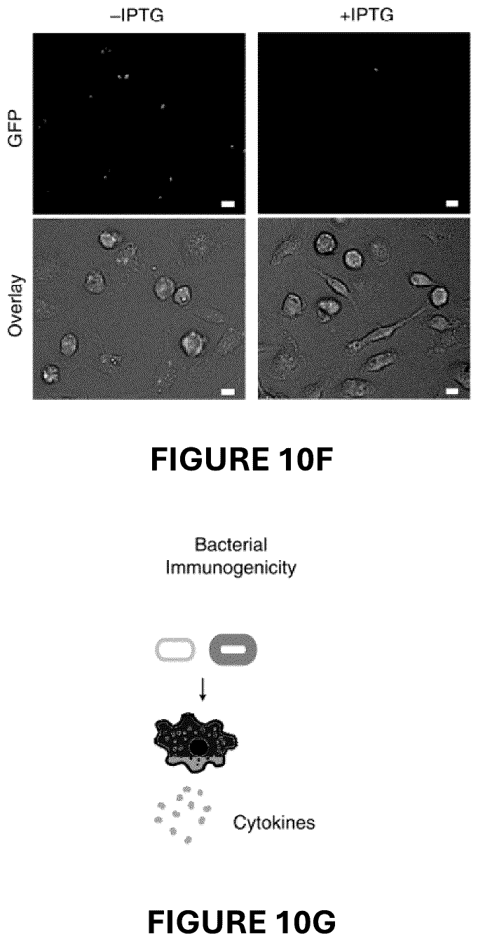

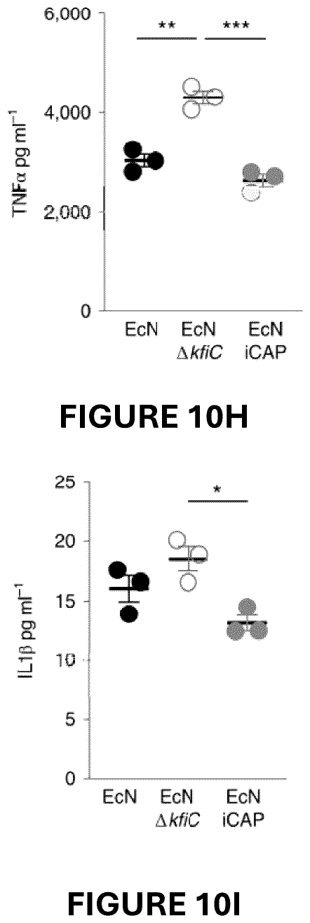

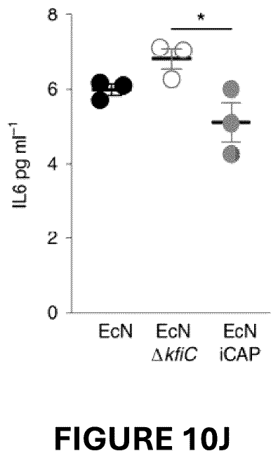

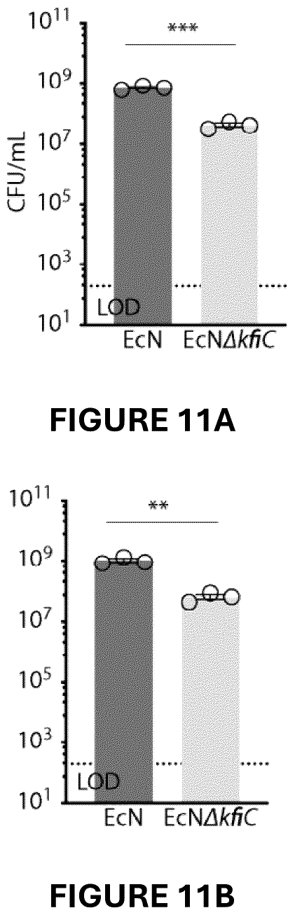

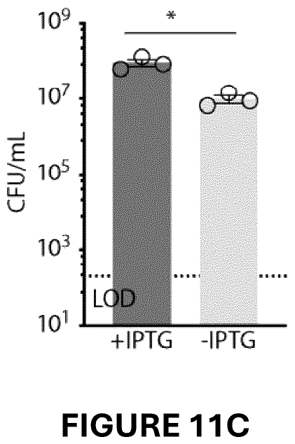

To build towards utilization of the programmable CAP system for therapeutic applications in vivo, exogenous control of bacterial viability in human whole blood containing functional host bactericidal factors was tested in vitro. Upon IPTG induction, increased EcN iCAP survival compared to non-induced control was observed (FIG. 10a). Increasing IPTG levels improved bacterial survival over a range of at least ˜10⁵ fold, highlighting the tunable capability of the system. Since iCAP deactivation was observed after removing the inducer, bacterial survival was measured over time after transiently activating EcN iCAP at varying IPTG concentration. Modulation of the rate of bacterial clearance from blood was possible by titrating levels of IPTG (FIG. 10b). Wildtype EcN persisted for >6 hours, while EcN ΔkfiC quickly decreased to the levels under the limit of detection (LOD ˜10² CFU/mL) within the first 0.5 hour. A protective role of CAP in mouse whole blood was also observed (FIGS. 11a and b).

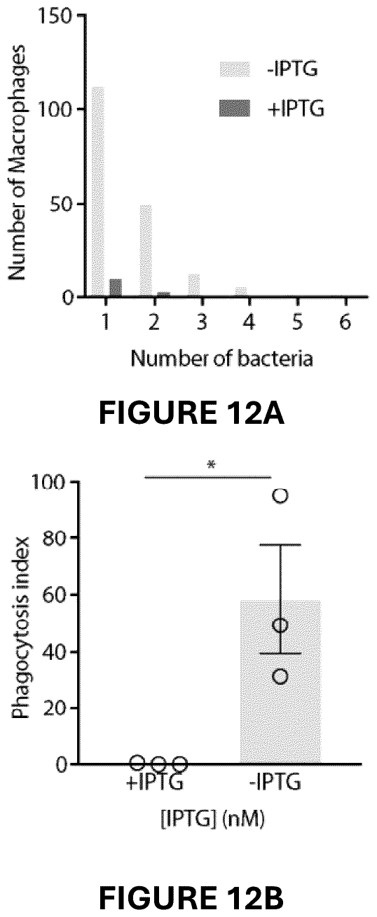

Autonomous systems that repress CAP expression upon sensing of specific conditions would be highly useful to clear bacteria and ensure safety. As a proof of principle, genetic circuits were designed to be capable of sensing (1) bacterial overgrowth at colonized sites, and (2) acidosis associated with sepsis to prevent systemic bacterial growth and inflammation. For both CAP systems, kfiC was placed under the control of tac promoter on the high (ColE1 origin) copy number plasmid to express CAP despite endogenous lacI expression. To design a CAP system responsive to bacterial overgrowth, a quorum-sensing module where bacteria express luxI gene to produce diffusible small molecule N-Acyl homoserine lactone (AHL). Upon reaching critical population density, the AHL-sensing pluxI promoter drives expression of lacI, repressing CAP production (FIG. 10c). The bacteria were cultured to stationary phase in LB media to simulate bacterial overgrowth, and observed that this quorum-sensing CAP (qCAP) system resulted in bacterial immunity against ΦK1-5. Control strains harboring a mutated luxI gene were sensitive to the phage, and exogenous addition of AHL molecule rescued bacterial immunity (FIG. 12a), confirming CAP repression via quorum-sensing circuit.

To test the safety feature of the system, bacteria were inoculated in human whole blood. Rapid elimination of the qCAP strain was observed after 2 hours while the control strain persisted (FIG. 10c), indicating bacterial overgrowth sensitizes bacteria to immune clearance via qCAP. To sense acidosis, a previously characterized pH-sensitive promoter pCadC was utilized. Membrane tethered endogenous CadC protein is cleaved to activate pCadC promoter in an acidic environment which drives expression of lac to repress CAP production (FIG. 10d). While the bacteria carrying this acidosis-sensing CAP (aCAP) system were sensitive to ΦK1-5 in a physiologically neutral condition (pH 7.4), they were able to grow in the presence of ΦK1-5 in a pH level similar to severe acidosis (pH 6.8) (FIG. 12b), indicating CAP repression upon sensing acidity. When the aCAP strain was inoculated in human whole blood, decreased levels of bacteria in acidic conditions were observed (FIGS. 10d and 12c). These genetic circuits highlight exogenous and autonomous control over bacterial CAP expression, allowing for programmable bacteria sensitivity to host immune detection to enhance safety.

To investigate the effect of the programmable CAP system on bacterial interaction with individual immune factors within whole blood, CAP alterations on modulated macrophage-mediated phagocytosis and complement-mediated killing were also studied.

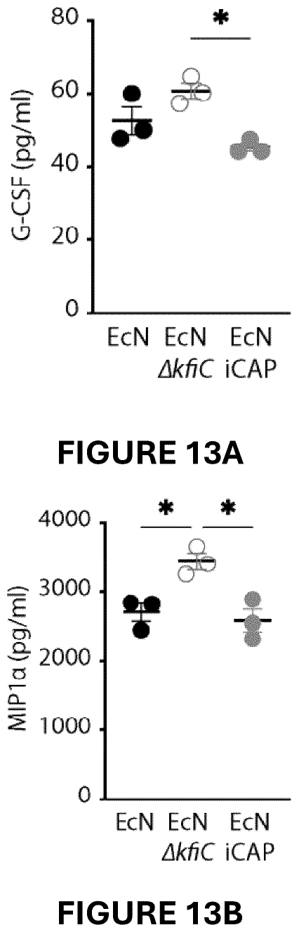

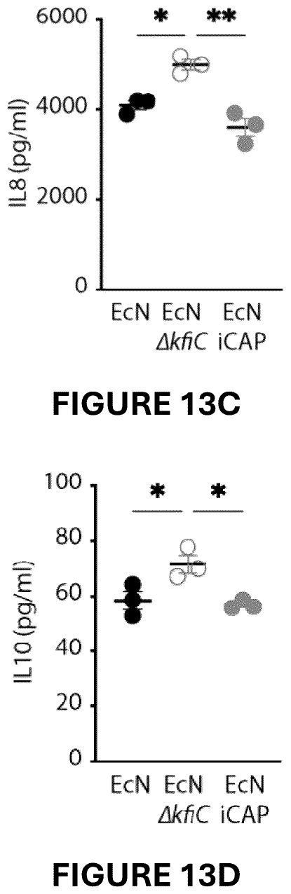

To study phagocytosis, EcN was incubated with murine bone marrow-derived macrophages. iCAP activation prior to co-incubation with macrophages resulted in reduction in uptake of bacteria within macrophages compared to basal control (FIG. 13 and b), demonstrating controllable protection from cellular immune recognition. Bacterial colony counting of macrophage lysates and fluorescence microscopy imaging confirmed ˜10-fold less phagocytosis with bacteria induced with IPTG compared to uninduced control (FIG. 10e). To assess inflammatory response by the phagocytes, EcN was co-cultured with THP-1 human monocytic cells and levels of TNFα were measured. Presence of CAP reduced levels of TNFα (FIG. 10f), indicating the ability of CAP to mask microbial recognition from the immune system.

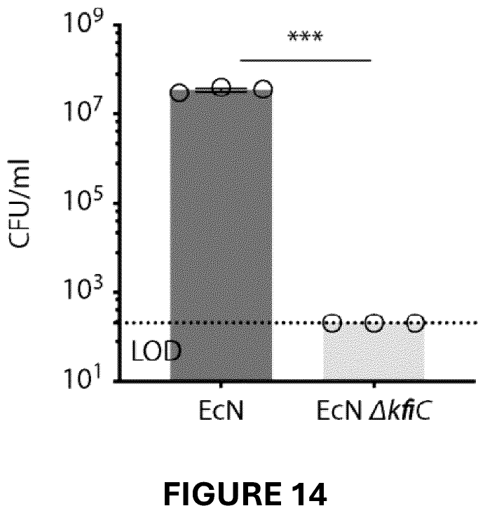

To study protection against circulating host antimicrobials such as the complement system, EcN was exposed to human plasma. The presence of CAP improved bacterial survival by at least ˜10⁵ fold (FIG. 14), demonstrating that CAP protects bacteria from soluble host bactericidal factors. Together, these findings suggest the potential utility of the programmable CAP system to modulates a multitude of host-microbe interactions in vivo.

### Example 18

### Transient CAP Improves Safety and Efficacy of Engineered Probiotic Therapy.

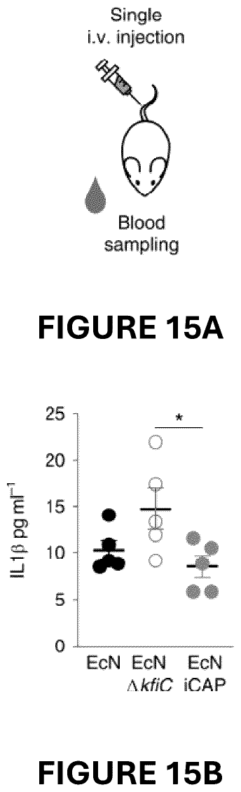

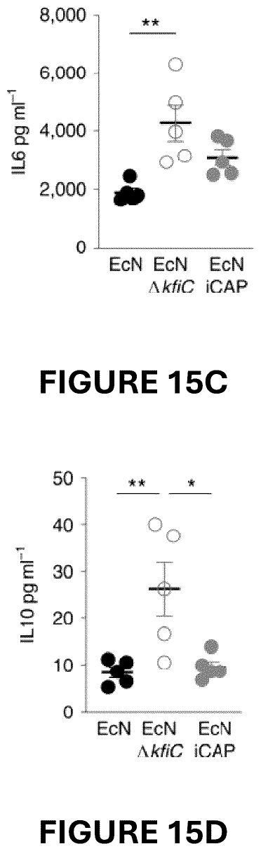

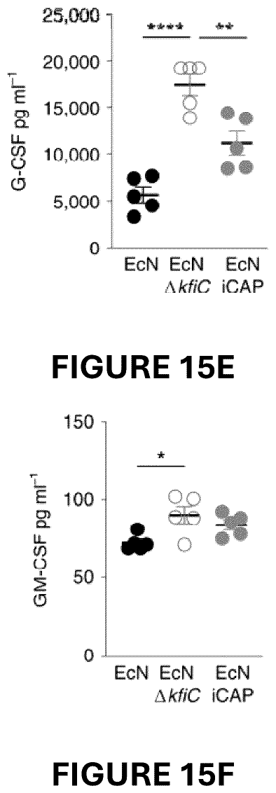

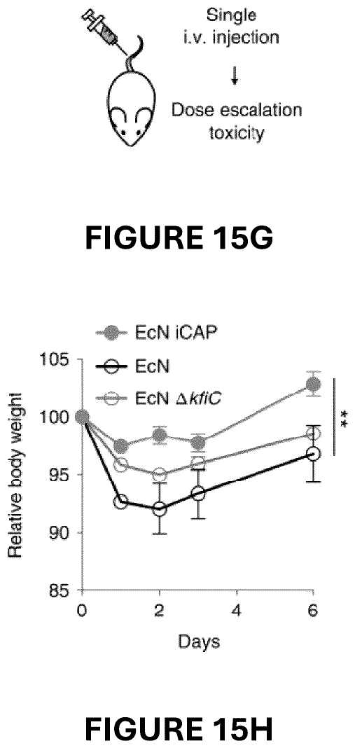

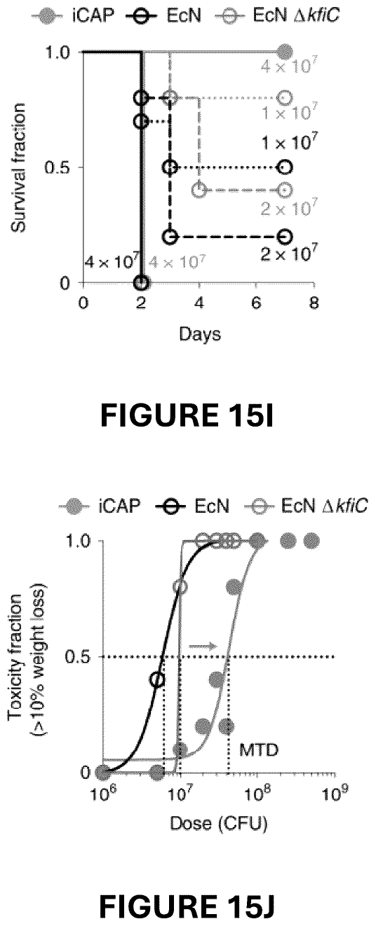

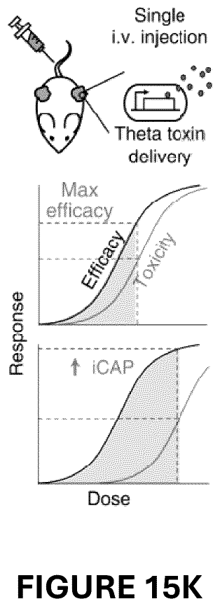

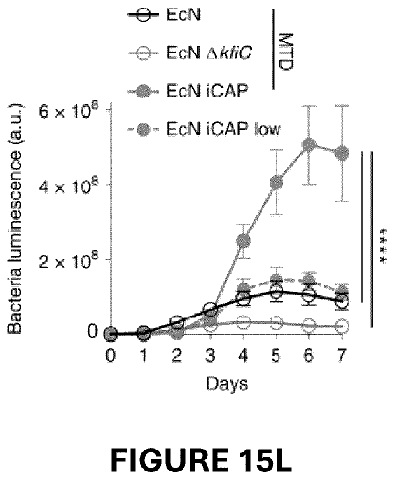

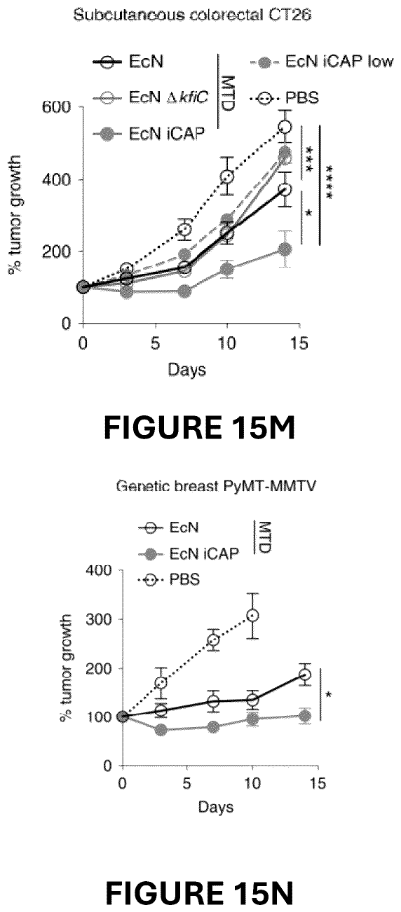

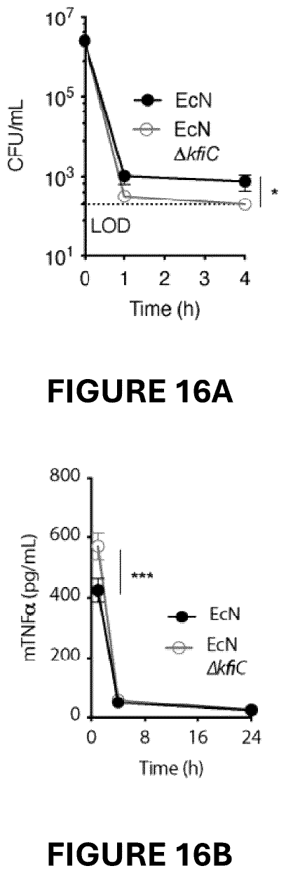

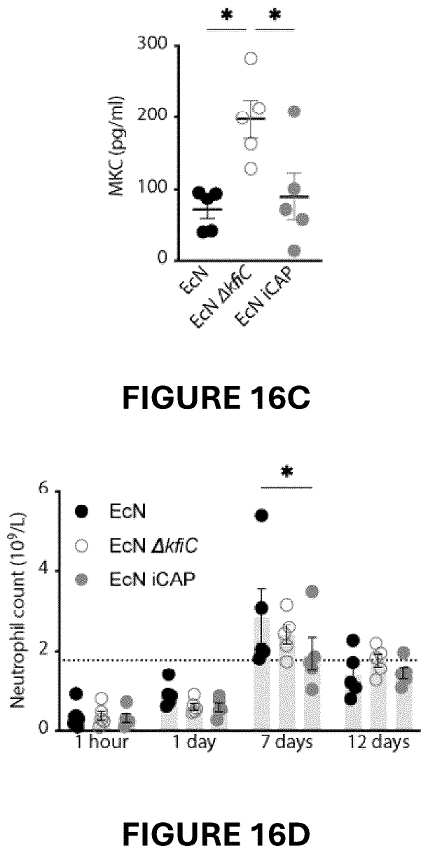

Intravenous (i.v.) delivery of bacteria allows access to various disease sites in the body; however, systemic delivery of bacteria remains challenging because (1) rapid clearance by the host immune system requires increased dosing, while (2) failure in bacteria clearance can lead to bacteremia and sepsis. To study the protective role of CAP in vivo, probiotic bioavailability and host health in mouse models was characterized (FIG. 15a). Upon i.v. administration of EcN ΔkfiC, viable bacteria in blood circulation quickly dropped below the LOD (200 CFU/mL). In contrast, EcN remained detectable during the first 4 hours (FIG. 16a), demonstrating the protective function of CAP in vivo. To examine the host response to encapsulated (i.e., wild type) vs. unencapsulated (i.e., ΔkfiC) EcN, levels of serum TNFα and total white blood cell count were measured. Lower levels of serum TNFα were detected in the first hour of EcN injection compared to EcN ΔkfiC injection (FIG. 15b), similar to the decreased TNFα response observed in in vitro assays. This short-term inflammatory response was resolved within 24 hours for both bacterial strains. In contrast to the rapid resolution of TNFα response, elevated total white blood cell count was detected after 24 hours of injection at higher level with EcN compared to EcN ΔkfiC (Supplementary FIG. 11b). Neutrophil expansion accounted for the majority of the immune response to encapsulated EcN (FIG. 15b), suggesting that the persistence of CAP-expressing EcN poses a risk of prolonged bacteremia, which may result in systemic inflammation and toxicity. Thus, while CAP can improve bioavailability, static protection may lead to prolonged bacterial circulation in blood and pose toxicity risks.

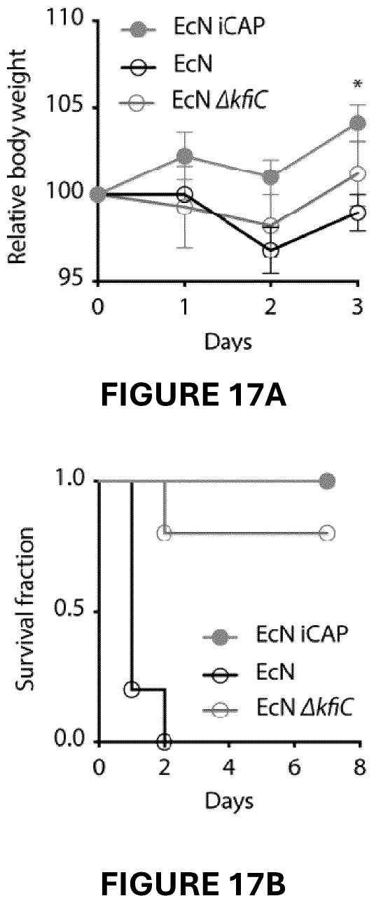

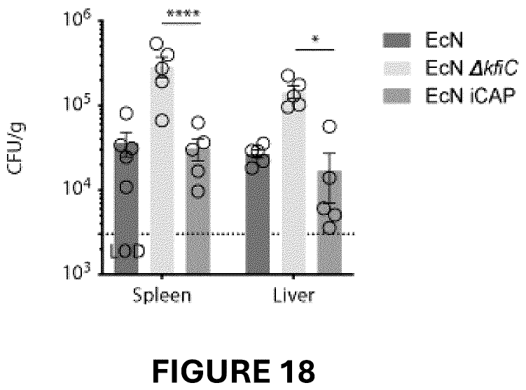

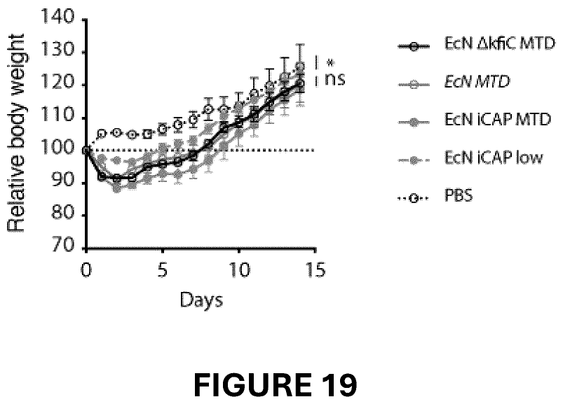

Transient activation of the programmable CAP system can improve bacterial delivery profiles by modulating maximum injectable dose, host toxicity, and biodistribution. Inducing CAP expression prior to injection would improve bioavailability and mask cytokine induction, and loss of CAP in the absence of the inducer in vivo would effectively clear bacteria and minimize long-term immune responses. To test this strategy, escalating doses of EcN iCAP were intravenously administered to mice and assessed host health and determined maximum tolerable dose (MTD) were assessed (FIG. 15c). At lower doses, EcN iCAP caused a smaller decrease in body weight compared to EcN and EcN ΔkfiC with static cellular surface (i.e., with or without CAP, respectively)(FIGS. 17a and b). Importantly, EcN iCAP dramatically reduced toxicity compared to EcN and EcN ΔkfiC at higher doses: EcN and EcN ΔkfiC caused severe end-point toxicity (death or >15% loss of weight) to mice treated with doses above 1×107 CFU within 2 days, while no mice showed severe toxicity following injection of pre-induced EcN iCAP at the same doses (FIG. 15d).

Based on these data, a dose-toxicity curve was generated. Transiently induced EcN iCAP results in ˜10-fold higher MTD compared to EcN and EcN ΔkfiC (FIG. 5e). To further study safety, a severe toxicity scenario was simulated by inducing sepsis by intraperitoneal injection of bacteria. At both high and low doses (107 and 10⁶ CFU, respectively), improved safety for EcN iCAP compared to EcN and EcN ΔkfiC was consistently observed (Supplementary FIG. 13a,b). Finally, to study bacteria biodistribution, all groups of mice were administered EcN at a matched dose of 5×10⁶ CFU via i.v. injection. Approximately 10-fold less EcN and EcN iCAP were found in peripheral organs (liver and spleen) compared to EcN ΔkfiC (Supplementary FIG. 14), indicating that initial induction of EcN iCAP was sufficient to provide protection from the mononuclear phagocyte system. These data support that transient activation of iCAP improves probiotic delivery and safety.

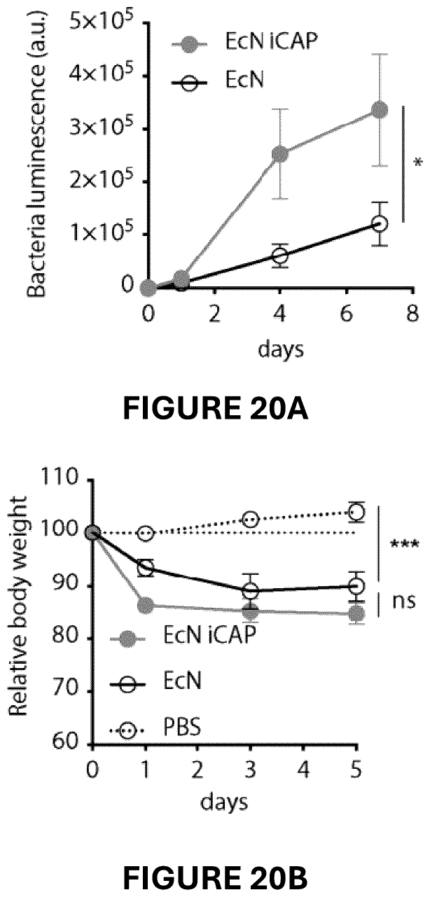

Since systemic bacterial delivery has been extensively used for cancer therapy, the effect of the programmable CAP system on improving antitumor efficacy was studied (FIG. 15f). To engineer bacteria to deliver antitumor payloads, a gene encoding a pore-forming toxin, theta toxin (TT), was cloned into a high copy number plasmid (ColE1) with a stabilization mechanism for in vivo applications (Axe/Txe system). In a syngeneic CT26 colorectal cancer model, engineered EcN were intravenously administered to mice at the corresponding MTD of each strain (EcN, EcN ΔkfiC, and EcN iCAP at 5×10⁶, 1×10⁷, and 5×10⁷ CFU, respectively), along with a low dose of EcN iCAP at 5×10⁶ CFU to match the MTD of EcN. Over the following days, bacterial accumulation in tumors was observed by luminescence. Here, EcN iCAP MTD showed significantly higher signals in tumors compared to all other groups (FIG. 15g). After bacterial administration, mice treated with EcN MTD, EcN ΔkfiC MTD, and low dose EcN iCAP exhibited modest tumor growth suppression compared to the untreated group over 14 days. By contrast, a single administration of EcN iCAP MTD resulted in significant tumor growth suppression by ˜400% compared to the untreated group (FIG. 15h). While increased MTD enabled by transient activation of the iCAP system improved therapeutic efficacy, body weight of animals between MTD groups remained similar (FIG. 20).

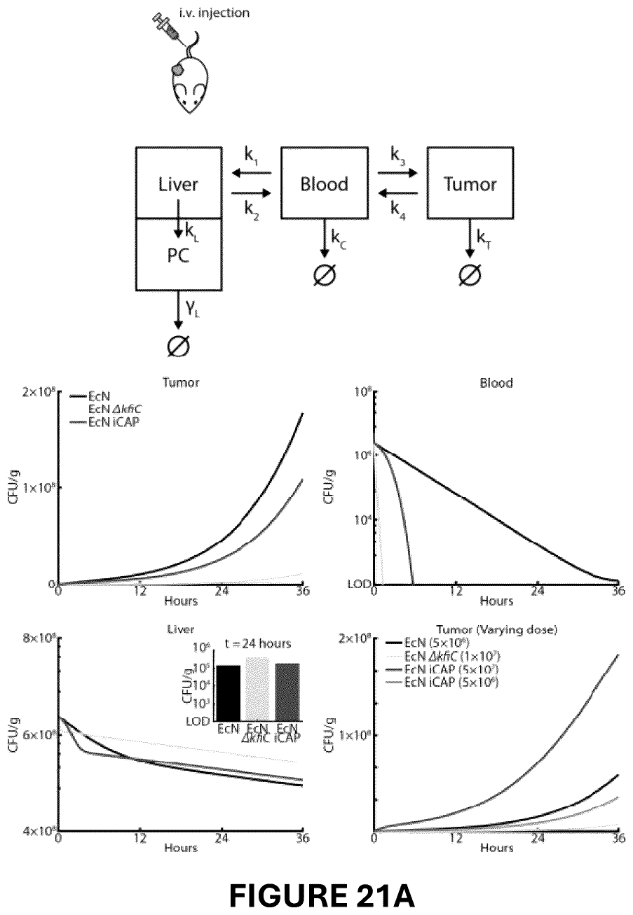

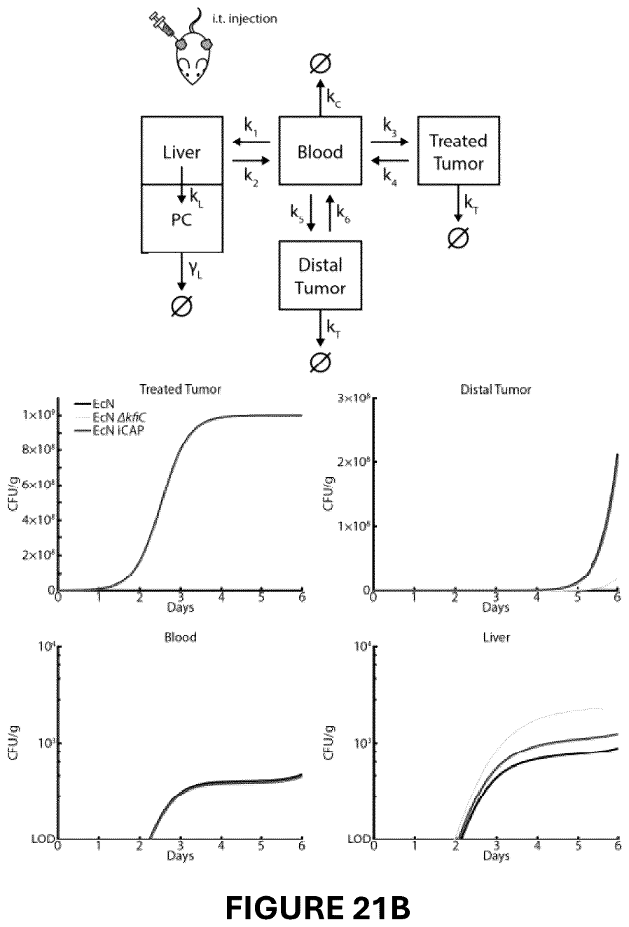

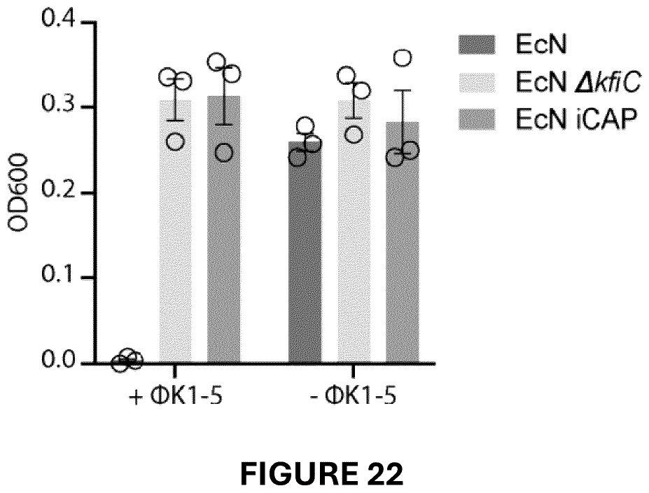

The efficacy of TT-producing EcN and EcN iCAP at MTD in a genetically engineered spontaneous breast cancer model (MMTV-PyMT) was also studied. EcN iCAP MTD resulted in improved tumor growth suppression by ˜100% compared to EcN MTD over 14 days (FIG. 15i). EcN iCAP consistently produced higher bacterial signal in tumors compared to EcN, while body weight remained similar between the two treatment groups (FIG. 21). To further explore the role of CAP on bacterial delivery to tumors in vivo, a mathematical bacterial pharmacokinetics model was produced. Simulations suggested transient protection of bacteria using the iCAP system could improve tumor specificity by minimizing persistence in peripheral organs (i.e., blood and liver), supporting these experimental observations (FIG. 22a). As a result, this approach allows for elevated bacterial doses, which leads to increased tumor accumulation upon injection. Taken together, the iCAP system enables increased tolerable bacterial doses and improved therapeutic efficacy.

### Example 19

### In Situ CAP Activation Translocates EcN to Distal Tumors

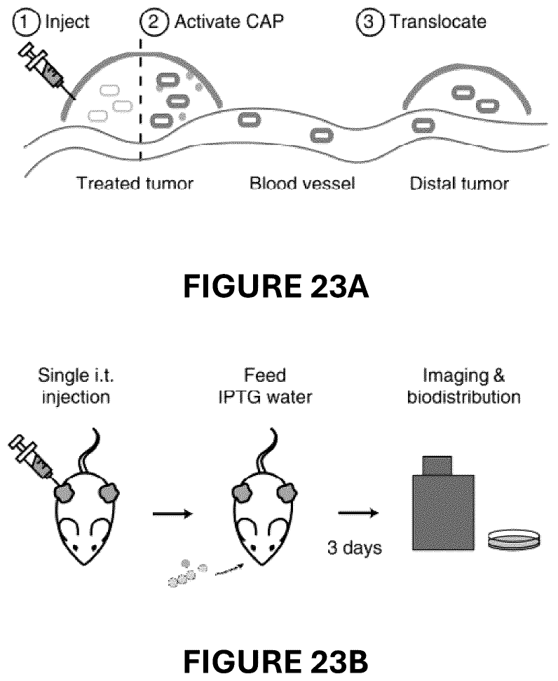

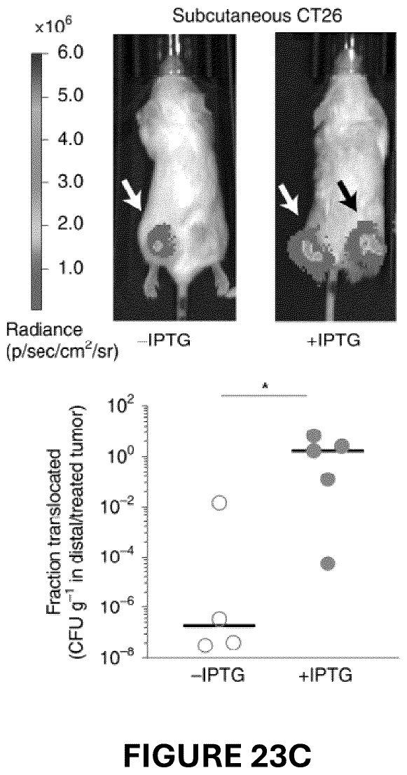

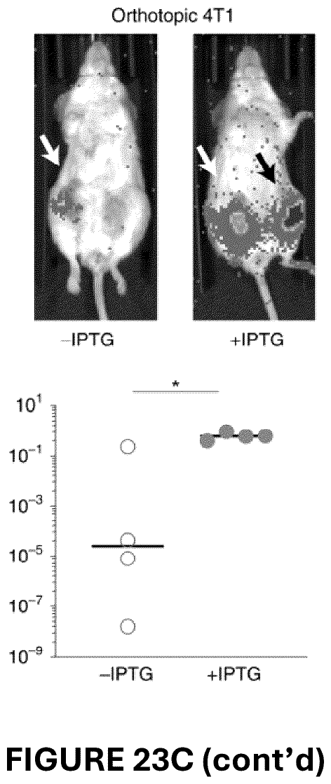

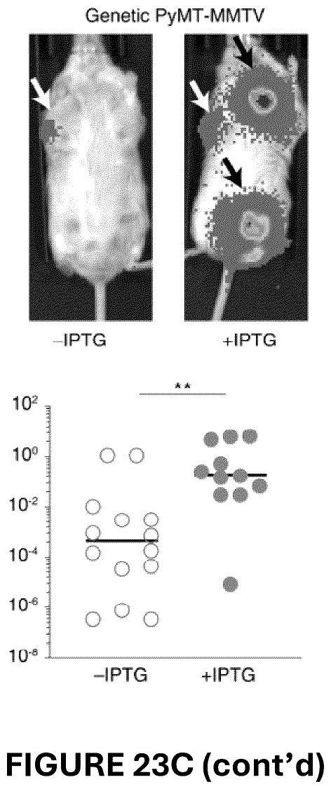

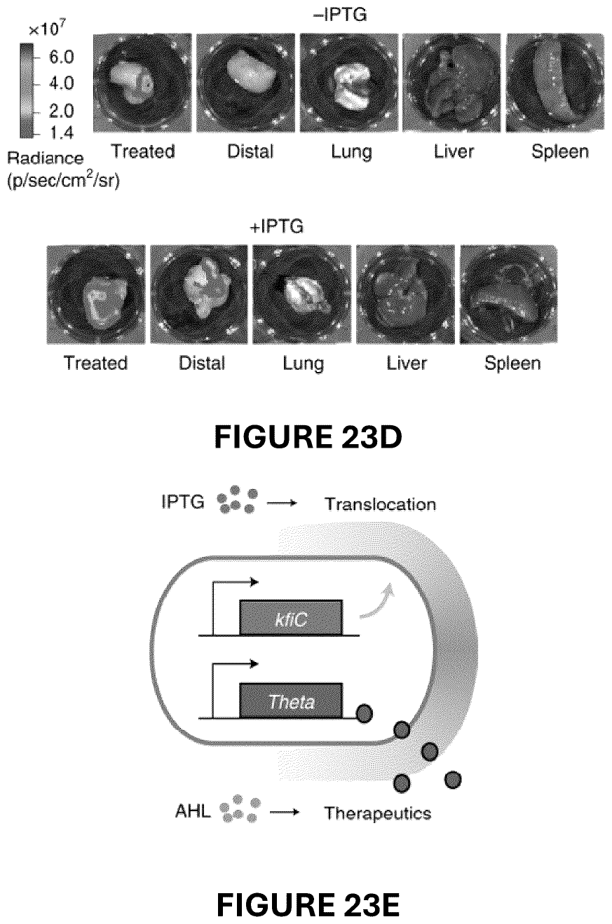

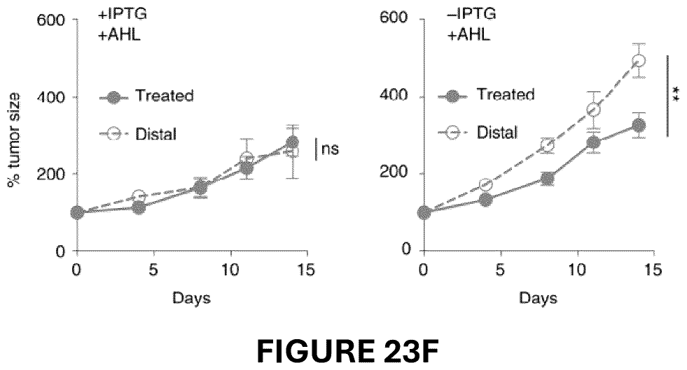

Intratumoral (i.t.) bacteria injection has been used as a route of delivery in clinical settings due to higher therapeutic efficacy, dose titration capability, and improved safety profiles compared to systemic injection. One unique capability of i.t. delivery is the translocation of bacteria from injected tumors to distal tumors, potentiating a novel route of safe bacterial delivery to inaccessible tumors. However, continuous translocation coupled with long-term survival of bacteria can pose a significant safety concern; thus, transient in situ activation could allow for more optimal utilization of this phenomena. In order to utilize this unique capability, simulated i.t. demonstrated that in situ induction of EcN iCAP within the tumor increases bacterial bioavailability in circulation and facilitates bacterial translocation to distal tumors (FIGS. 22b and 23a).

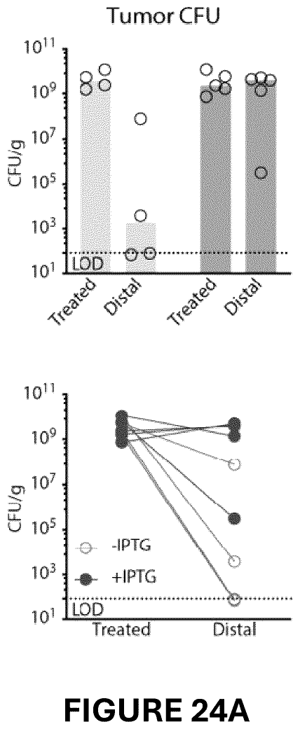

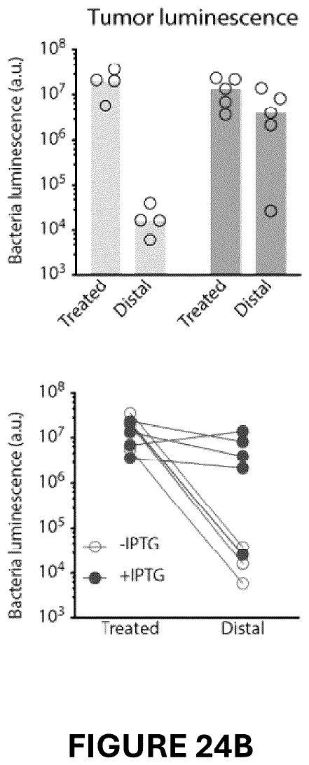

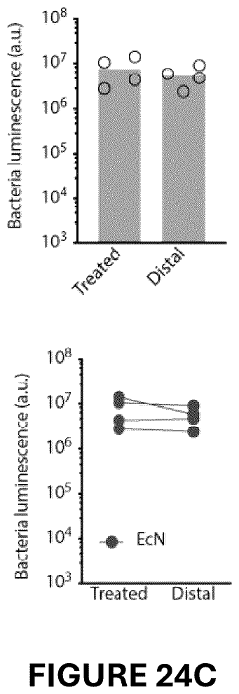

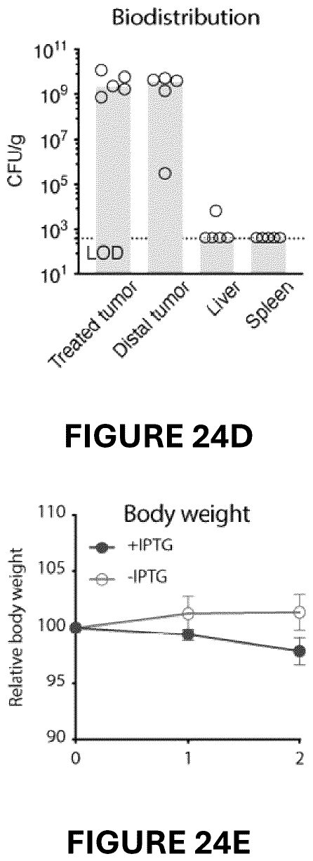

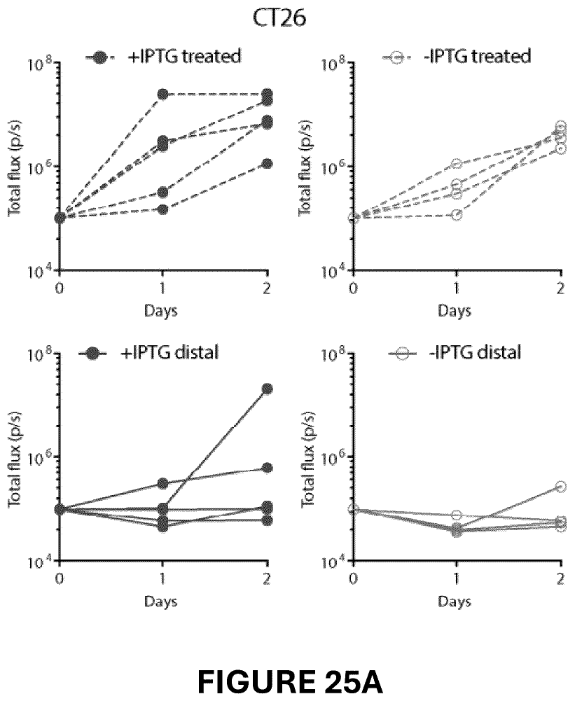

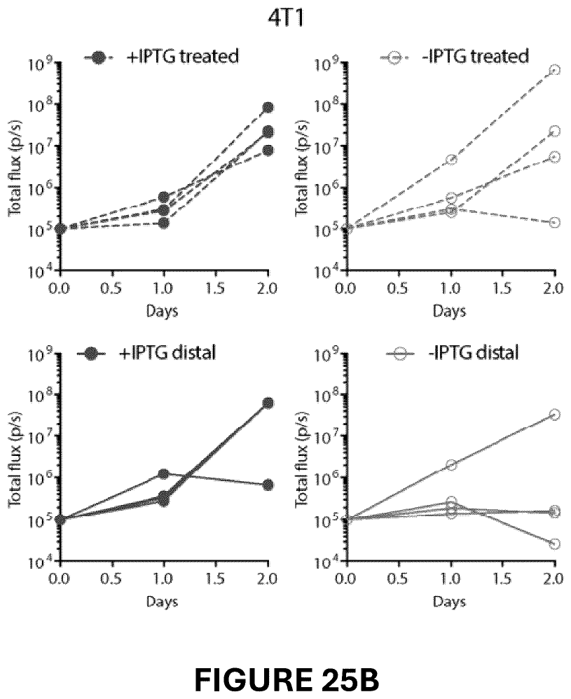

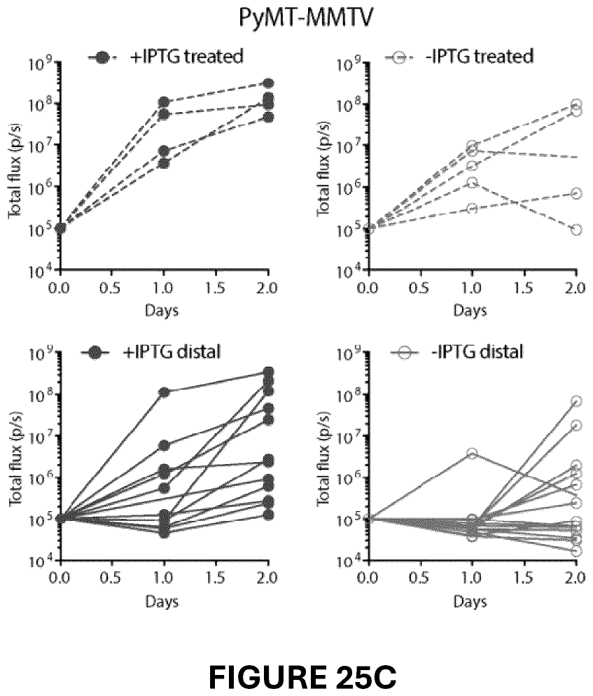

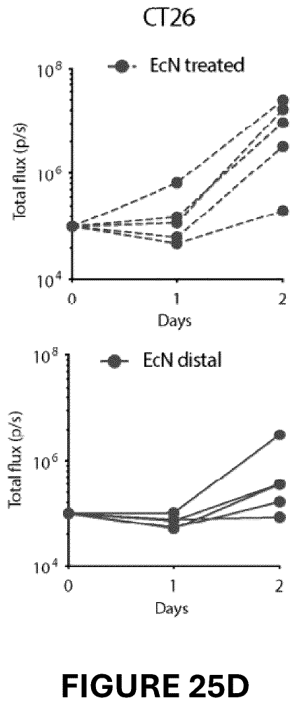

Intratumoral injection of uninduced EcN iCAP (i.e., without CAP) into a single tumor of mice harboring dual hind-flank CT26 tumors was subsequently carried out (FIG. 23b). To activate the iCAP system in situ, mice were fed with water containing IPTG. After 3 days, a marked increase in bacterial translocation to distal tumors compared to uninduced bacteria was observed (FIGS. 23c and 24a,b). Biodistribution data showed tumor-specific translocation (FIG. 23d, FIG. 24c), and mice exhibited minimal reductions in body weight (FIG. 24). Tracing colonization kinetics using bioluminescent EcN confirmed the appearance of bacteria in distal tumor 1 day following IPTG administration (FIG. 25).

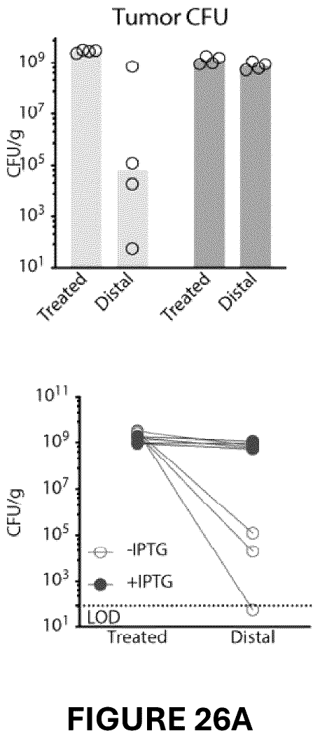

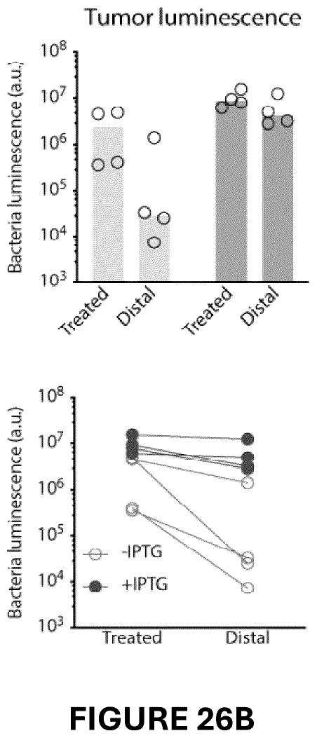

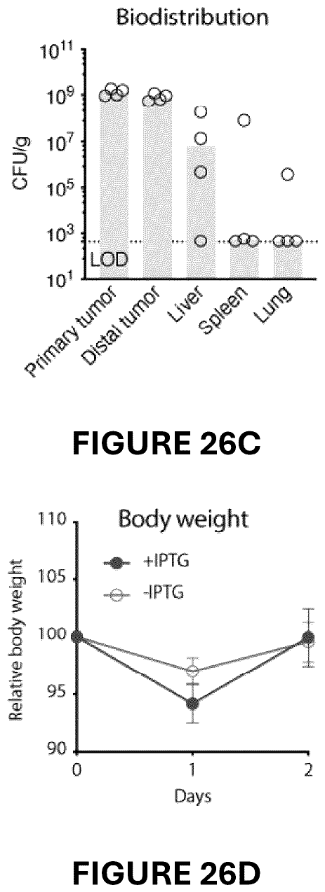

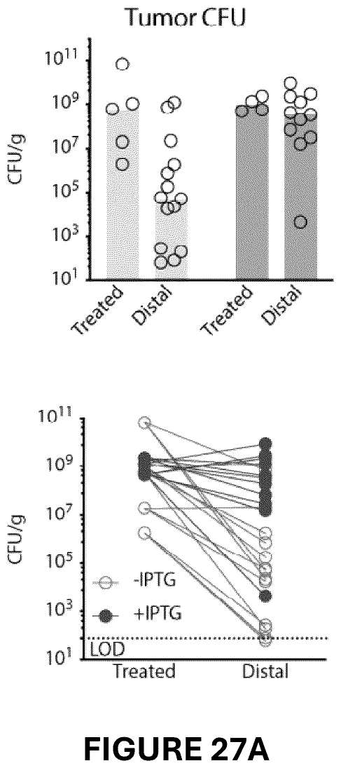

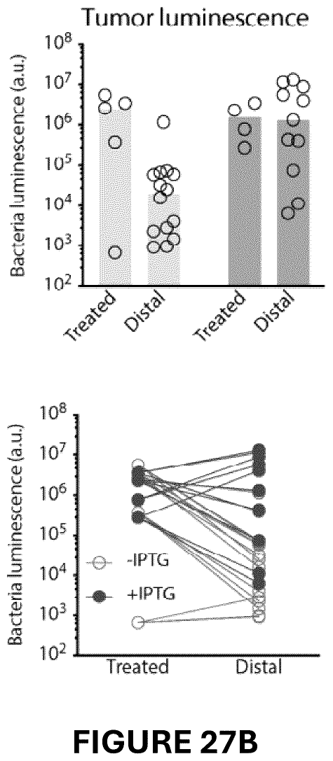

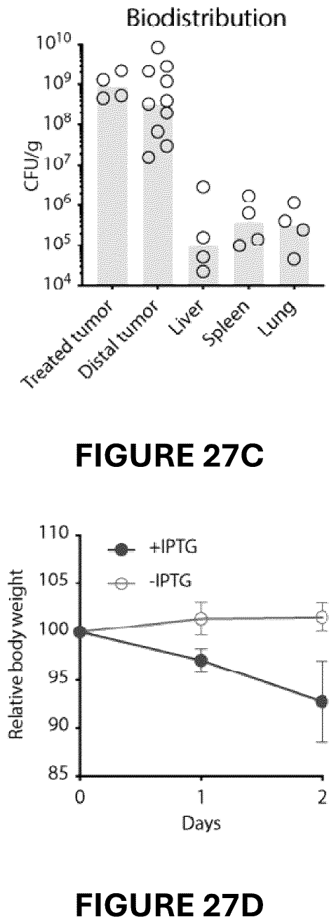

Orthotopic breast cancer (mammary fat-pad 4T1) and MMTV-PyMT mouse models were also tested. Consistently, we observed increased bacterial translocation to distal tumors via in situ activation of iCAP in both tumor models (FIGS. 2, 23, 26 and 27). Notably, i.t. injection of EcN iCAP into a single tumor in the MMTV-PyMT model resulted in microbial translocation to multiple distal tumors throughout the body following IPTG induction. These results demonstrate the robustness of iCAP-mediated translocation across a range of locations and tumor types.

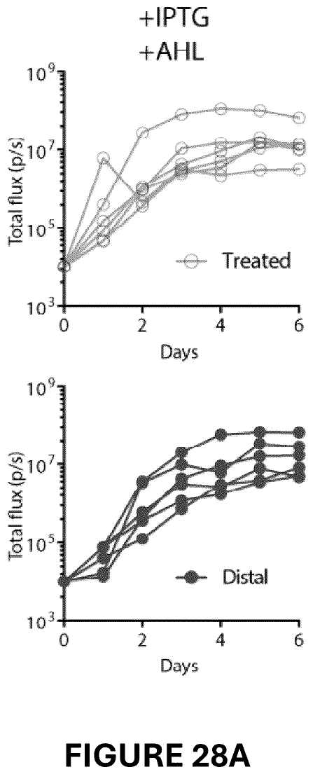

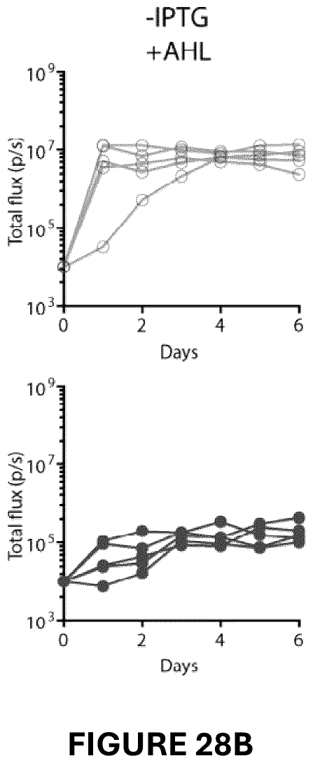

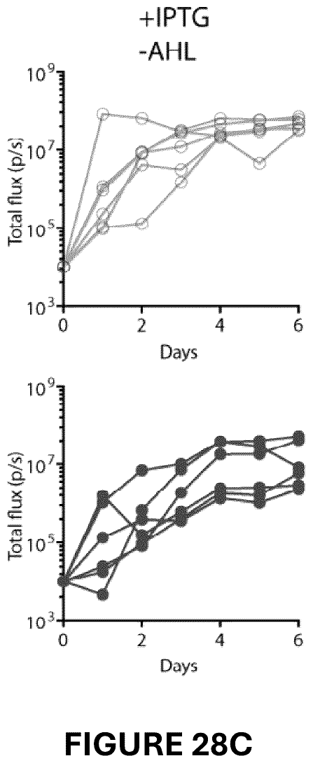

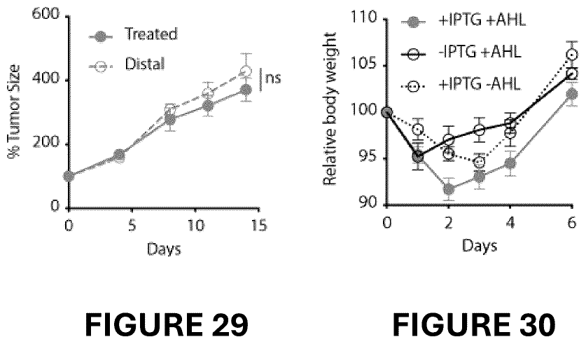

Therapeutics were delivered to tumors using engineered EcN expressing the antitumor TT payload. TT was cloned under the luxI promoter that is responsive to an inducer molecule AHL orthogonal to IPTG (FIG. 6e). Following i.t. injection of therapeutic EcN into a single tumor in the CT26 dual flank mouse model, translocation to uninjected tumors was controlled by feeding mice with or without IPTG water. Another group of mice were given i.t. injection of EcN iCAP without TT as non-therapeutic control. iCAP-mediated bacterial translocation was confirmed by bacterial bioluminescence (FIG. 28). Subsequently, AHL was administered subcutaneously to induce TT expression, and tumor growth was monitored. While PBS treatment allowed tumor growth of ˜400%, a reduction in the growth of tumors were observed when they were directly injected with therapeutic EcN regardless of bacterial translocation. By contrast, therapeutic efficacy in distal (uninjected) tumors was only observed when the mice were fed with IPTG water followed by TT induction via subcutaneous injection of AHL (FIGS. 23f and 29), demonstrating successful therapeutic delivery to distal tumors using the iCAP-mediated bacterial translocation approach. Body weight quickly recovered to baseline within a few days post administration for all conditions (FIG. 30). Together, this demonstrates controllably translocating therapeutic bacteria and utilizing this strategy to treat distal tumors in vivo.

While this invention has been disclosed with reference to particular embodiments, it is apparent that other embodiments and variations of the inventions disclosed herein can be devised by others skilled in the art without departing from the true spirit and scope thereof. The appended claims include all such embodiments and equivalent variations.

What is claimed is:

1. A programmable bacterium, comprising a gene which regulates capsular polysaccharide nanoencapsulation of the bacterium linked to an exogenous promoter, wherein expression of the gene and the nanoencapsulation can be programmed or controlled by an external modulator of the exogenous promoter.

2. The programmable bacterium of claim 1, wherein the bacterium is E. coli.

3. The programmable bacterium of claim 1, wherein the bacterium is E. coli Nissle 1917 bacteria.

4. The programmable bacterium of claim 1, wherein the gene that regulates capsular polysaccharide nanoencapsulation is chosen from the group consisting of kfi and kps genes.

5. The programmable bacterium of claim 1, wherein the gene that regulates capsular polysaccharide nanoencapsulation is chosen from the group consisting of kfiA, kfiB, kfiC, kfiD, kpsE, kpsD, kpsM, kpsT, kpsC, kpsS, kpsF and kpsU.

6. The programmable bacterium of claim 1, wherein the exogenous promoter is lac and the external modulator is isopropyl-b-D-thiogalactopyranoside (IPTG).

7. The programmable bacterium of claim 1, wherein the exogenous promoter is plux1 and the external modulator is N-acyl homoserine lactone (AHL).

8. The programmable bacterium of claim 1, wherein the exogenous promoter is pCadC and the external modulator is pH.

9. The programmable bacterium of claim 1, further comprising at least one plasmid comprising a nucleic acid sequence that encodes a therapeutic agent.

10. The programmable bacterium of claim 9, wherein the nucleic acid sequence encoding the therapeutic agent is under control of a second inducible exogenous promoter.

11. The programmable bacterium of claim 10, wherein the second inducible exogenous promoter is different than the exogenous promoter operably linked to the gene that regulates capsular polysaccharide nanoencapsulation.

12. A pharmaceutical composition comprising the programmable bacterium of claim 1 and one or more pharmaceutically acceptable carriers, diluents, or excipients.

13. A method of treating cancer in a subject in need thereof comprising administering to the subject a therapeutically effective amount of the programmable bacterium of claim 1.

14. The method of claim 13, wherein the programmable bacterium is administered in the form of a pharmaceutical formulation.

15. The method of claim 13, wherein the cancer is colorectal cancer or breast cancer.

16. The method of claim 13, wherein the programmable bacterium is administered to the subject intratumorally, orally, intravenously, subcutaneously, or intratumorally.

17. The method of claim 13, further comprising inducing the gene which regulates capsular polysaccharide nanoencapsulation of the programmable bacterium linked to an exogenous promoter upon administration of the bacterium to the subject and ceasing induction upon the programmable bacterium entering a target tumor, organ, or tissue.
